(19) United States
(12) Patent Application Publication
(10) Pub. No.: US 2013/0028933 A1
(43) Pub. Date: Jan. 31, 2013

(54) METHODS FOR STABILIZING INFLUENZA ANTIGEN ENVELOPED VIRUS-BASED VIRUS-LIKE PARTICLE SOLUTIONS

(71) Applicants: Joel R. Haynes, Bozeman, MT (US); Bryan Steadman, Bozeman, MT (US)

(72) Inventors: Joel R. Haynes, Bozeman, MT (US); Bryan Steadman, Bozeman, MT (US)

(73) Assignee: LigoCyte Pharmaceuticals, Inc., Bozeman, MT (US)

(21) Appl. No.: 13/517,240

(22) PCT Filed: Dec. 28, 2010
(86) PCT No.: PCT/US10/62217

§ 371 Date: Jun. 19, 2012

Related U.S. Application Data
(60) Provisional application No. 61/290,438, filed on Dec. 28, 2009.

(51) Int. Cl. C07K 1/02; A61P 31/16; A61P 37/04; A61K 39/145; C07K 14/11

(52) U.S. Cl. 424/210.1; 530/350; 530/396; 424/209.1

(57) ABSTRACT

Methods of stabilizing solutions with enveloped vims-based virus-like particles containing an influenza antigen and such stabilized solutions are described.

### FIELD

The present invention relates to the field of stabilized enveloped virus-based virus-like particles that include an influenza antigen. In particular, methods of stabilizing such virus-like particles and stabilized compositions comprising such virus like-particles are disclosed herein.

### BACKGROUND

Influenza. A and B are the two types of influenza viruses that cause epidemic human disease (111). Influenza A viruses are further categorized into subtypes on the basis of two surface antigens: hemaggalutinin (HA) and neuraminidase (NA). Influenza B viruses are not categorized into subtypes, but do under go drift whereby strains diverge over time. Since 1977, influenza. A (H1N1) viruses, influenza A (H3N2) viruses, and influenza B viruses have been in global circulation. Influenza A (H1N2) viruses that probably emerged after genetic reassortment between human A (H3N2) and A (H1N1) viruses have been detected recently in many countries. Both influenza A and B viruses are further separated into groups on the basis of antigenic characteristics. New influenza virus variants result from frequent antigenic change (i.e., antigenic drift) resulting from point mutations that occur during viral replication. Influenza B viruses undergo antigenic drift less rapidly than influenza A viruses. Frequent development of antigenic variants through antigenic drift is the virologic basis for seasonal epidemics and the reason for the incorporation of at least one new strain in each year's influenza vaccine.

A person's immunity to the surface antigens, especially hemagglutinin, reduces the likelihood of infection and severity of disease if infection occurs (112). It is generally thought that antibody against one influenza virus type or subtype confers limited or no protection against another. Furthermore, it is generally accepted that antibody to one antigenic variant of influenza virus might not protect against a new antigenic variant of the same type or subtype (113). Therefore, the demonstration of cross-protection is unexpected.

Human-avian reassortant influenza viruses were responsible for the previous two influenza pandemics in 1957 and 1968. Since H2 viruses have not circulated in humans after 1968, an antigenic shift arising from an H2 reassortant virus is theoretically possible at any time. However, the recent emergence of highly pathogenic avian influenza (HPAI) viruses (H5 and H7) and the sporadic transmission of these viruses directly from birds to humans since 1997 (1-5) brings a new human pandemic threat potential in addition to the population's ever increasing susceptibility to H2 viruses. The fact that human HPAI H5N1 outbreaks have been antigenically distinct makes it all but impossible to prepare advance stockpiles of a well-matched vaccine against a pandemic threat (5, 6). While mouse H5 immunization and challenge data indicate that good cross reactivity is seen between various H5 isolates in this model (7), it is not known if similar levels of cross reactivity will be seen in humans with existing vaccine technology. Thus, there is a need for influenza vaccine platforms that may be quickly adapted to include antigens from new viral outbreaks.

The present egg-based inactivated vaccine technology is inadequate to meet the demands of an emerging pandemic due to the inability to propagate HPAI viruses in eggs and the need for enhanced biocontainment (6, 8). Reverse genetics approaches offer a means of producing low pathogenicity reassortants with the desired HA and NA makeup that can be cultured in eggs (7, 9-11); however, vaccines produced by this approach are only now entering the clinic due to previous intellectual property and regulatory issues (8). An additional concern is the apparent low level inummogenicity associated with H5 hemagglutinins evaluated in human clinical trials (12-14) which makes it clear that improved vaccines, delivery systems, and the use of adjuvants may be required to efficiently induce protection in a population that is completely H5-naïve. Thus, there is a need for an influenza vaccine platform that allows for expression of HPAI antigens in combination with adjuvants.

Influenza VLPs represent an alternative technology for generating influenza vaccines. Influenza VLPs have been produced using the influenza matrix, HA and NA proteins expressed in insect cells which are markedly immunogenic following intranasal delivery (26, 27). Indeed, VLPs in general appear well suited for the induction of mucosal and systemic immunity following intranasal delivery as has been shown for rotavirus, norovirus, and papilloma virus VLPs (28-31). Influenza VLPs have been produced in eukaryotic expression systems by expression of influenza matrix, HA and NA proteins. The influenza matrix is the driving force behind virus budding and NA is required for budded VLP release from producer cells when HA is also being expressed owing to HA's association with sialic acid at the cell surface (51). There are also data to indicate that interactions between matrix and the C-terminus of HA play a role in directing matrix to the membrane as part of the budding process (51). Influenza VLPs produced in an insect cell baculovirus expression system have proven immunogenic in animal trials and represent an important strategy for future pandemic preparedness (26, 27, 47). In addition, intranasal delivery of influenza VLPs can result in antibody titers exceeding those obtained following parenteral administration. However, VLPs are complex structures of lipid membranes embedded with one or more different glycoproteins embedded in or associated with the membrane. To have practical utility, the VLPs will need to have a reasonably long shelf-life. Thus, there is a need for methods of stabilizing virus like particles that include an influenza antigen in a liquid solution.

### SUMMARY

The disclosure of the present application meets this need by providing methods of stabilizing virus like particles that include an influenza antigen in a liquid solution together with the stabilized solutions and methods of using such stabilized solutions.

One aspect of the disclosure provides methods for stabilizing a solution containing an influenza antigen enveloped virus-based virus-like particle preparation comprising (a) providing the solution containing the influenza antigen enveloped virus-based virus-like particle; and (b) (1) adding a stabilizing amount of a stabilizing agent selected from a monosaccharide, sorbitol, a disaccharide, trehalose, diethanolamine, glycerol, glycine, and a combination of the preceding stabilizing agents to influenza antigen enveloped virus-based virus-like particle preparation, (2) buffering the solution so that the pH is between about pH 6.5 and about pH 8.0, between about pH 6.5 and about pH 7.5, or about pH 7, or (3) both steps (1) and (2), wherein the influenza antigen enveloped virus-based virus-like particle preparation after step (b) exhibits at least one of the following characteristics (i) reduced aggregation of the virus-like particles as compared to the influenza antigen enveloped virus-based virus-like particle preparation before step (b) as measured by optical density, (ii) stabilized influenza antigen as compared to the influenza antigen enveloped virus-based virus-like particle preparation before step (b) as measured by circular dichroism or ANS binding, and (iii) reduced temperature induced hydration of the lipid bilayer of the virus-like particle as compared to the influenza antigen enveloped virus-based virus-like particle preparation before step (b) as measured by laurdan fluorescence. In certain embodiments, the buffering is performed using a buffering agent selected from the group consisting of phosphate, Tris, MES, citrate and other GRAS buffers. In certain embodiments, which may be combined with the preceding buffering agent embodiments, the stabilizing agent is selected from trehalose, sorbitol, diethanolamine, glycerol, glycine and a combination of the preceding stabilizing agents and the characteristic is (i). In certain embodiments, which may be combined with the preceding buffering agent embodiments, the stabilizing agent is selected from trehalose, sorbitol, and a combination of the preceding stabilizing agents and the characteristic is (ii). In certain embodiments, which may be combined with the preceding buffering agent embodiments, the stabilizing agent is selected from trehalose and glycine and the characteristic is (iii). In certain embodiments, which may be combined with the preceding buffering agent embodiments, the stabilizing agent is trehalose and all three characteristics are present. In certain embodiments, which may be combined with any of the preceding embodiments, the influenza antigen enveloped virus-based virus-like particle comprises a hemagglutinin polypeptide. In certain embodiments, which may be combined with any of the preceding embodiments, the influenza antigen enveloped virus-based virus-like particle comprises a second polypeptide selected from the group comprising a gag polypeptide, an influenza M1 polypeptide, a Newcastle disease virus matrix polypeptide, an Ebola virus VP40 polypeptide and a Marburg virus VP40 polypeptide. In certain embodiments, which may be combined with any of the preceding embodiments including a gag polypeptide, the gag polypeptide may be from murine leukemia virus, human immunodeficiency virus, Alpharetroviruses, Betaretroviruses, Gammaretroviruses, Deltaretroviruses and Lentiviruses. In certain embodiments, which may be combined with any of the preceding embodiments, the influenza antigen enveloped virus-based virus-like particle further comprises a neuraminidase polypeptide. In certain embodiments, which may be combined with any of the preceding embodiments, the stabilizing agent is selected from monosaccharide, sorbitol, a disaccharide, and trehalose, and the stabilizing amount is greater than 10% (w/w) or at least about 20% (w/w). In certain embodiments, which may be combined with any of the preceding embodiments, the stabilizing does not require glass formation. In certain embodiments, which may be combined with any of the preceding embodiments, the stabilizing amount is less that the amount required for glass formation upon freezing. In certain embodiments, which may be combined with any of the preceding embodiments, the stabilizing agent is not sucrose. In certain embodiments, which may be combined with any of the preceding embodiments, the influenza antigen enveloped virus-based virus-like particle preparation further comprises an adjuvant in admixture with the influenza antigen enveloped virus-based virus-like particle, which may be located inside said virus-like particle or located outside said virus-like particle. In certain embodiments, which may be combined with any of the preceding embodiments that include an adjuvant and a second polypeptide, the adjuvant is covalently linked to the second polypeptide to form a covalent linkage. In certain embodiments, which may be combined with any of the preceding embodiments that include an adjuvant and a hemagglutinin polypeptide, the adjuvant is covalently linked to said hemagglutinin polypeptide to form a covalent linkage. In certain embodiments, which may be combined with any of the preceding embodiments that include an adjuvant, the adjuvant comprises an adjuvant-active fragment of flagellin. In certain embodiments, which may be combined with any of the preceding embodiments, the methods further comprise step (c) storing the solution in liquid form for a period of time of at least two weeks, at least one month, at least two months, at least three months, at least four months, at least six months, or at least one year, wherein the influenza antigen enveloped virus-based virus-like particle preparation after such time period induces at least eighty percent, at least ninety percent, or at least ninety five percent of the immune response induced by the influenza antigen enveloped virus-based virus-like particle preparation before such time period.

Another aspect of the disclosure provides an influenza antigen enveloped virus-based virus-like particle preparations comprising influenza antigen enveloped virus-based virus-like particles and a stabilizing amount of a stabilizing agent selected from trehalose, sorbitol, diethanolamine, glycerol, glycine, and a combination of the preceding stabilizing agents to influenza antigen enveloped virus-based virus-like particle preparation, wherein the influenza antigen enveloped virus-based virus-like particle preparation exhibits at least one of the following characteristics (i) reduced aggregation of the virus-like particles as compared to a influenza antigen enveloped virus-based virus-like particle preparation without the stabilizing agent as measured by optical density, (ii) stabilized influenza antigen as compared to the influenza antigen enveloped virus-based virus-like particle preparation without the stabilizing agent as measured by circular dichroism or ANS binding, and (iii) reduced temperature induced hydration of the lipid bilayer of the virus-like particle as compared to the influenza antigen enveloped virus-based virus-like particle preparation without the stabilizing agent as measured by laurdan fluorescence. In certain embodiments, the buffering is performed using a buffering agent selected from the group consisting of phosphate, Tris, MES, citrate and other GRAS buffers. In certain embodiments, the stabilizing agent is selected from trehalose, sorbitol, diethanolamine, glycerol, glycine and a combination of the preceding stabilizing agents and the characteristic is (i). In certain embodiments, the stabilizing agent is selected from trehalose, sorbitol, and a combination of the preceding stabilizing agents and the characteristic is (ii). In certain embodiments, the stabilizing agent is selected from trehalose and glycine and the characteristic is (iii). In certain embodiments, the stabilizing agent is trehalose and all three characteristics are present. In certain embodiments, which may be combined with any of the preceding embodiments, the influenza antigen enveloped virus-based virus-like particle comprises a hemagglutinin polypeptide. In certain embodiments, which may be combined with any of the preceding embodiments, wherein the influenza antigen enveloped virus-based virus-like particle comprises a second polypeptide selected from the group comprising a gag polypeptide, an influenza M1 polypeptide, a Newcastle disease virus matrix polypeptide, an Ebola virus VP40 polypeptide and a Marburg virus VP40 polypeptide. In certain embodiments, which may be combined with any of the preceding embodiments that include a gag polypeptide, the gag polypeptide is from a retrovirus selected from the group consisting of murine leukemia virus, human immunodeficiency virus, Alpharetroviruses, Betaretroviruses, Gammaretroviruses, Deltaretroviruses and Lentiviruses. In certain embodiments, which may be combined with any of the preceding embodiments, the influenza antigen enveloped virus-based virus-like particle further comprises a neuraminidase polypeptide. In certain embodiments, which may be combined with any of the preceding embodiments, the stabilizing agent is selected from monosaccharide, sorbitol, a disaccharide, and trehalose, and the stabilizing amount is greater than 10% (w/w) or at least about 20% (w/w). In certain embodiments, which may be combined with any of the preceding embodiments, the stabilizing does not require glass formation. In certain embodiments, which may be combined with any of the preceding embodiments, the stabilizing amount is less that the amount required for glass formation upon freezing. In certain embodiments, which may be combined with any of the preceding embodiments, the stabilizing agent is not sucrose. In certain embodiments, which may be combined with any of the preceding embodiments, the influenza antigen enveloped virus-based virus-like particle preparation further comprises an adjuvant in admixture with the influenza antigen enveloped virus-based virus-like particle. In certain embodiments, which may be combined with any of the preceding embodiments that include an adjuvant, the adjuvant is located inside said virus-like particle. In certain embodiments, which may be combined with any of the preceding embodiments that include an adjuvant, the adjuvant is located outside said virus-like particle. In certain embodiments, which may be combined with any of the preceding embodiments that include an adjuvant and a second polypeptide, the adjuvant is covalently linked to said second polypeptide to form a covalent linkage. In certain embodiments, which may be combined with any of the preceding embodiments that include an adjuvant, adjuvant is covalently linked to said hemagglutinin polypeptide to form a covalent linkage. In certain embodiments, which may be combined with any of the preceding embodiments, the adjuvant comprises an adjuvant-active fragment of flagellin.

Another aspect of the disclosure provides methods for treating or preventing influenza comprising administering to a subject an iummnogenic amount of the influenza antigen enveloped virus-based virus-like particle preparation of the preceding aspect and any of its various embodiments or a solution containing an immunogenic amount of an influenza antigen enveloped virus-based virus-like particle preparation stabilized in accordance with the preceding method aspect and any of its various embodiments. In certain embodiments, the administering induces a protective immunization response in the subject. In certain embodiments that may be combined with the preceding embodiments, the administering is selected from the group consisting of subcutaneous delivery, transcutaneous delivery, intradermal delivery, subdermal delivery, intramuscular delivery, peroral delivery, oral delivery, intranasal delivery, buccal delivery, sublingual delivery, intraperitoneal delivery, intravaginal delivery, anal delivery and intracranial delivery.

### SUMMARY OF THE FIGURES

FIG. 1 shows western blots of the media from Sf9 cells infected with separate Gag, HA or control vectors and with HA-gag-NA triple vectors. (A) was probed with anti-Gag antibodies and (B) was probed with anti-HA antibodies.

FIG. 2 shows western blots of fractions from a sucrose step gradient recentrifugation of pelleted HA-gag-NA VLPs. (A) was probed with anti-Gag antibodies and (B) was probed with anti-HA antibodies.

FIG. 3 shows the dynamic light scattering by influenza VLPs. Effective diameter (A), static light scattering intensity (B), and sample polydispersity (C) are plotted as a function of temperature. Each point represents the mean of three independent samples, and error bars show the standard deviation.

FIG. 4 shows the circular dichroism spectra of influenza VLPs. Low temperature (10° C.) spectra at each unit pH from 4 to 8 (A) and pH 7 spectra at a variety of temperatures (B) are shown.

FIG. 5 shows the response of influenza VLP protein secondary structure to thermal stress. The normalized (−1 to 0) CD at 227 nm is presented as a function of temperature. Each point represents the mean of three independent samples, and error bars show the standard deviation.

FIG. 6 shows the intrinsic fluorescence peak position of influenza VLPs as a function of temperature. Also presented as a function of temperature (lower right) is the normalized (0 to 1) intensity of fluorescence at 330 nm. Each point represents the mean of three independent samples, and error bars show the standard deviation.

FIG. 7 shows the fluorescence of ANS as a probe of influenza VLP physical structure. The wavelength of peak emission is presented as a function of temperature. Also presented as a function of temperature (lower right) is the normalized (0 to 1) intensity of ANS fluorescence at 485 nm. Each point represents the mean of three independent samples, and error bars show the standard deviation.

FIG. 8 shows a generalized polarization of laurdan fluorescence in the presence of influenza VLPs as a function of temperature. Each point represents the mean of three independent samples, and error bars show the standard deviation.

FIG. 9 shows the empirical phase diagram derived from biophysical characterization of influenza VLPs. The EPD is prepared from temperature-dependent effective diameter, static light scattering, polydispersity, CD at 227 nm, intrinsic fluorescence (peak position and relative intensity at 330 nm), ANS fluorescence (Peak position and relative intensity at 485 nm), and GP of laurdan fluorescence data collected across the pH range from 4 to 8.

FIG. 10 shows the intrinsic fluorescence of influenza VLPS in the presence of selected stabilizers. The position (wavelength) of the peak emission is presented as a function of temperature. Each point represents the mean of two independent samples, and error bars show the standard deviation.

FIG. 11 shows the GP of laurdan fluorescence in the presence of influenza VLPs formulated with selected stabilizers. Each point represents the mean of two independent samples, and error bars show the standard deviation.

### DETAILED DESCRIPTION OF THE PREFERRED EMBODIMENTS

The present invention is based upon stabilized formulations of influenza antigen enveloped-virus based VLPs. An exemplary enveloped-virus based VLP includes gag polypeptides as the basis for formation of the VLPs, such as the gag polypeptide from the murine leukemia virus (MLV). Au exemplary method of generating such gag-based VLPs is by expression in insect cells, preferably including coexpression of an influenza HA and an NA polypeptide antigen, because of the significant yields of gag VLPs that can be obtained from a variety of retroviruses in the baculovirus expression system.

The stabilization is mediated by a stabilizing amount of a stabilizing agent that is included with the influenza antigen-virus based VLP preparation. Exemplary stabilizing agents include monosaccharides (such as dextrose, mannitol, sorbitol) disaccharides (such as lactose, trehalose, sucrose) diethanolamine, glycerol, glycine, or combinations thereof.

The practice of the disclosed methods and protocols will employ, unless otherwise indicated, conventional techniques of chemistry, molecular biology, microbiology, recombinant. DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### DEFINITIONS

An “enveloped virus-based VLP” as used here refers to virus-like particles that are formed using one or more components derived from an enveloped virus. Preferred examples include, without limitation, VLPs generated using gag polypeptides with influenza hemagglutinin polypeptides, VLPs generated using influenza hemagglutinin polypeptides or Orthomyxovirus (including influenza) M1 polypeptides with hemagglutinin polypeptides (in each case optionally with neuraminidase polypeptides), VLPs generated using Paromyxovirus (including Newcastle disease virus) matrix polypeptides with influenza hemagglutinin polypeptides, and VLPs generated using Filovirus (including Ebola or Marburg virus) VP40 polypeptides with influenza hemagglutinin polypeptides.

Additional examples include: filoviruses such as Ebola virus and Marburg virus may be used to form enveloped virus based VLPs (e.g., coexpression of virus GP and VP40 from filoviruses in cells will generate VLPs owing to the association of these two viral proteins in lipid rafts (see U.S. Pat. Publ. 20060099225)); coronaviruses such as SARS (e.g., E and M proteins are sufficient for coronavirus VLP formation (see Fischer et al., J. Virol. (1998) 72:7885-7894 and Vennema et al. EMBO J. (1996) 15:2020-2028)); paramyxoviridae viruses such as respiratory syncytial virus (RSV) (e.g., expression of the M protein of RSV will generate VLPs (See, e.g., U.S. Pat. Publ. 20080233150)); and flaviviridae such as West Nile Virus (e.g., expressing a construct comprising the prM and E genes of a West Nile Virus in baculovirus expression system will generate VLPs (See, e.g., U.S. Pat. Publ. 20080233150)).

Gag polypeptides include the retrovirus derived structural polypeptide that is responsible for formation of the virus like particles described herein. In some embodiments, the gag polypeptide may be purposely mutated in order to affect certain characteristics such as the propensity to package RNA or the efficiency of particle formation and budding. One example of such a mutation would be amino acid changes that affect the ability of gag-derived VLPs to incorporate RNA. Other such amino acid changes could be made that improve or modify the efficiency of VLP budding. The genome of retroviruses codes for three major gene products: the gag gene coding for structural proteins, the poi gene coding for reverse transcriptase and associated proteolytic polypeptides, nuclease and integrase associated functions, and env whose encoded glycoprotein membrane proteins are detected on the surface of infected cells and also on the surface of mature released virus particles. The gag genes of all retroviruses have an overall structural similarity and within each group of retroviruses are conserved at the amino acid level. The gag gene gives rise to the core proteins excluding the reverse transcriptase. For MLV the Gag precursor polyprotein is Pr65^{Gag} and is cleaved into four proteins whose order on the precursor is NH₂-p15-pp12-p30-p10-COOH. These cleavages are mediated by a viral protease and may occur before or after viral release depending upon the virus. The MLV Gag protein exists in a glycosylated and a non-glycosylated form. The glycosylated forms are cleaved from gPr80^{Gag} which is synthesized from a different inframe initiation codon located upstream from the AUG codon for the non-glycosylated Pr65^{Gag}. Deletion mutants of MLV that do not synthesize the glycosylated Gag are still infectious and the non-glycosylated Gag can still form virus-like particles, thus raising the question over the importance of the glycosylation events. The post translational cleavage of the HIV-1 Gag precursor of pr55^{Gag} by the virus coded protease yields the N-myristoylated and internally phosphorylated p17 matrix protein (p17MA), the phosphorylated p24 capsid protein (p24CA), and the nucleocapsid protein p15 (p15NC), which is further cleaved into p9 and p6.

Structurally, the prototypical Gag polyprotein is divided into three main proteins that always occur in the same order in retroviral gag genes: the matrix protein (MA) (not to be confused with influenza matrix protein M1, which shares the name matrix but is a distinct protein from MA), the capsid protein (CA), and the nucleocapsid protein (NC). Processing of the Gag polyprotein into the mature proteins is catalyzed by the retroviral encoded protease and occurs as the newly budded viral particles mature. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. The form of the Gag protein that mediates assembly is the polyprotein. Thus, the assembly domains need not lie neatly within any of the cleavage products that form later. The Gag polypeptide as included herein therefore includes the important functional elements for formation and release of the VLPs. The state of the art is quite advanced regarding these important functional elements. See, e.g., Hansen et al. J. Virol 64, 5306-5316, 1990; Will et al., AIDS 5, 639-654, 1991; Wang et al. J. Virol. 72, 7950-7959, 1998; McDonnell et al., J. Mol. Biol. 279, 921-928, 1998; Schultz and Rein, J. Virol. 63, 2370-2372, 1989; Accola at al., J. Virol. 72, 2072-2078, 1998; Borsetti et al., J. Virol., 72, 9313-9317, 1998; Bowzard et al., J. Virol. 72, 9034-9044, 1998; Krishna et al., J. Virol. 72, 564-577, 1998; Wills et al., J. Virol. 68, 6605-6618, 1994; Xiang et al., J. Virol. 70, 5695-5700, 1996; Garnier et al., J. Virol. 73, 2309-2320, 1999.

Exemplary retroviral sources for Gag polypeptides include murine leukemia virus, human immunodeficiency virus, Alpharetroviruses (such as the avian leucosis virus or the Rous sarcoma virus), Betaretroviruses (such as mouse mammary tumor virus, Jaagsiekte sheep retrovirus and Mason-Phizer monkey virus), Gammaretroviruses (such as murine leukemia virus, feline leukemia virus, reticuloendotheliosis virus and gibbon ape leukemia virus), Deltaretroviruses (such as human T-lymphotrophic virus and bovine leukemia virus), Epsilonretroviruses (such as walleye dermal sarcoma virus), or Lentiviruses (human immunodeficiency virus type 1, HIV-2, simian immunodeficiency virus, feline immunodeficiency virus, equine infectious anemia virus, and caprine arthritis encephalitis virus).

The “hemagglutinin polypeptide” as used herein is derived from the influenza virus protein that mediates binding of the virus to the cell to be infected. The protein is an antigenic glycoprotein found anchored to the surface of influenza viruses by a single membrane spanning domain. At least sixteen subtypes of the influenza hemagglutinin have been identified labeled H1 through H16. H1, H2, and H3, are found in human influenza viruses. Highly pathogenic avian flu viruses with H5, H7 or H9 hemagglutinins have been found to infect humans at a low rate. It has been reported that single amino acid changes in the avian virus strain's type H5 hemagglutinin have been found in human patients that alters the receptor specificity to allow the H5 hemagglutinin to significantly alter receptor specificity of avian H5N1 viruses, providing them with an ability to bind to human receptors (109 and 110). This finding explains how an H5N1 virus that normally does not infect humans can mutate and become able to efficiently infect human cells.

Hemagglutinin is a homotrimeric integral membrane polypeptide. The membrane spanning domain naturally associates with the raft-lipid domains, which allows it to associate with the gag polypeptides for incorporation into VLPs. It is shaped like a cylinder, and is approximately 135 Å long. The three identical monomers that constitute HA form a central coiled-coil and a spherical head that contains the sialic acid binding sites, which is exposed on the surface of the VLPs. HA monomers are synthesized as a single polypeptide precursor that is glycosylated and cleaved into two smaller polypeptides: the HA1 and HA2 subunits. The HA2 subunits form the trimeric coiled-coil that is anchored to the membrane and the HA1 subunits form the spherical head.

As used in the VLPs of the present invention, the hemagglutinin polypeptide shall at a minimum include the membrane anchor domain and at least one epitope from hemagglutinin. The hemagglutinin polypeptide may be derived from any influenza virus type, subtype, strain or substrain, such as from the H1, H2, H3, H5, H7 and H9 hemagglutinins. In addition, the hemagglutinin polypeptide may be a chimera of different influenza hemagglutinins. The hemagglutinin polypeptide may optionally include one or more additional polypeptides that may be generated by splicing the coding sequence for the one or more additional polypeptides into the hemagglutinin polypeptide coding sequence. An exemplary site for insertion of additional polypeptides into the hemagglutinin polypeptide is the N-terminus.

The “neuraminidase polypeptide” as used herein is derived from the influenza virus protein that mediates release of the influenza virus from the cell by cleavage of terminal sialic acid residues from glycoproteins. The neuraminidase glycoprotein is expressed on the viral surface. The neuraminidase proteins are tetrameric and share a common structure consisting of a globular head with a beta-pinwheel structure, a thin stalk region, and a small hydrophobic region that anchors the protein in the virus membrane by a single membrane spanning domain. The active site for sialic acid residue cleavage includes a pocket on the surface of each subunit formed by fifteen charged amino acids, which are conserved in all influenza A viruses. At least nine subtypes of the influenza neuraminidase have been identified labeled N1 through N9.

As may be used in the VLPs disclosed herein, the neuraminidase polypeptide shall at a minimum include the membrane anchor domain and at least the sialic acid residue cleavage activity. The state of the art regarding functional regions is quite high. See, e.g., Varghese et al., Nature 303, 35-40, 1983; Colman et al., Nature 303, 41-44, 1983; Lentz et al., Biochem, 26, 5321-5385, 1987; Webster et al., Virol. 135, 30-42, 1984. The neuraminidase polypeptide may be derived from any influenza virus type, subtype strain or substrain, such as from the N1 and N2 neuraminidases. In addition, the neuraminidase polypeptide may be a chimera of different influenza neuraminidase. The neuraminidase polypeptide may optionally include one or more additional polypeptides that may be generated by splicing the coding sequence for the one or more additional polypeptides into the neuraminidase polypeptide coding sequence. An exemplary site for insertion of additional polypeptides into the neuraminidase polypeptide is the C-terminus.

The “GRAS buffers” as used herein refers to buffers that are “generally recognized as safe” as announced by an applicable governmental regulatory agency. GRAS buffers preferably will buffer within the range of pH 6 to pH 8 (e.g., pKa 5-9). The effective buffering range of a compound is generally accepted to be pKa±about 1 pH unit, e.g., buffering capacity for H₃PO₄ with a pKa of 7.2 is approximately 6.5-8.0. Exemplary GRAS buffers will include tris(hydroxymethyl)aminomethane (Tris), hydrogen or dihydrogen citrate, monobasic potassium phosphate, dibasic potassium phosphate, monobasic sodium phosphate, dibasic sodium phosphate, 2-(N-morpholino)ethauesulfonic acid (MES), 3-(N-morpholino)propanesulfonic acid (MOPS), N-(2-Acetamido)iminodiacetic Acid (ADA), 3-[N-Tris(hydroxymethyl)methylamino]-2-hydroxypropanesulfonic Acid (TAPSO), 3-[N,N-bis(2-Hydroxyethyl)amino]-2-hydroxypropanesulfonic Acid (DIPSO), piperazine-N,N′-bis(2-hydroxypropanesulfonic acid) (POPSO), N-(2-Hydroxymethyl) piperazine-N′-2-hydroxypropanesulfonic acid (HEPPSO), piperazine-N,N′-bis(2-ethanesulfonic acid) (PIPES), N-(2-Acetamido)-2-aminoethanesulfonic acid (ACES), Cholamine chloride, N,N-Bis(2-hydroxyethyl)-2-aminoethanesulfonic Acid (BES), N-Tris(hydroxymethyl)methyl-2-aminoethanesulfonic Acid (TES), (4-(2-hydroxyethyl)-1-piperazineethanesulfonic acid) (HEPES), Acetamidoglycine, tricine, glycinamide, bicine, amino acid residues such as Histidine, acetate, ammonium hydroxide, imidazole, 2-amino-2-methyl-1-propanol (AMP), 2-amino-2-methyl-1,3-propanediol (AMPD), 2-[Bis(2-hydroxyethyl)amino]-2-(hydroxymethyl)-1,3-propanediol (BIS-Tris), 1,3-bis(tris(hydroxymethyl)methylamino)propane (BIS-Tris propane), carbonate, citrate, ethanolamine, glycylglycine; N-(2-Hydroxyethyl)piperazine-N(4-butanesulfonic acid) (HEPBS), and maleic acid. Exemplary functional groups that can provide buffering in the appropriate range include imidazole, carboxylic acids, phosphates, piperazine, amines, and sulfonic acid. While GRAS is generally defined for use in food, GRAS buffers as used herein also includes buffers that have been deemed pharmaceutically acceptable for inclusion in drug formulations.

Exemplary Methods of Making Enveloped Virus-Based VLPs

Enveloped virus-based VLPs may be made by any method available to one of skill in the art. Enveloped virus-based VLPs typically include one or more polypeptide responsible for the formation of the VLP in addition to the influenza antigen polypeptide, which includes chimeric forms where the polypeptide responsible for formation of the VLP is itself an influenza antigen such as M1 or is linked to the influenza antigen polypeptide. In addition, the enveloped virus-based VLP may include one or more additional polypeptide such as a membrane (including lipid-raft)-associated polypeptide to provide additional antigens such as a second influenza antigen or another antigen. In certain embodiments, the polypeptides may be co-expressed in any available protein expression system, such as a cell-based system that includes lipid raft domains in the plasma membrane such as mammalian cell expression systems and insect cell expression systems.

Recombinant expression of the polypeptides for the VLPs involves expression vectors containing polynucleotides that encode one or more of the polypeptides. Once a polynucleotide encoding one or more of the polypeptides has been obtained, the vector for the production of the polypeptide may be produced by recombinant DNA technology using techniques well known in the art. Thus, methods for preparing a protein by expressing a polynucleotide containing any of the VLP polypeptide-encoding nucleotide sequences are described herein. Methods which are well known to those skilled in the art can be used to construct expression vectors containing the VLP polypeptide coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a gag polypeptide and a lipid-raft associated polypeptide linked to antigen, all operably linked to one or more promoters.

The expression vector may be transferred to a host cell by conventional techniques and the transfected cells are then cultured by conventional techniques to produce the VLP polypeptide(s). Thus, the invention includes host cells containing a polynucleotide encoding one or more of the VLP polypeptides operably linked to a heterologous promoter. In certain embodiments for the generation of VLPs, vectors encoding both the gag polypeptide and a lipid-raft associated polypeptide linked to an influenza antigen (or the influenza antigen itself may be a lipid-raft associated polypeptide) may be co-expressed in the host cell for generation of the VLP, as detailed below.

A variety of host-expression vector systems may be utilized to express the VLP polypeptides. Such host-expression systems represent vehicles by which the VLP polypeptides may be produced to generate VLPs such as by co-expression. A wide range of hosts may be used in construct of appropriate expression vectors and, when relying upon lipid-raft based assembly, preferred host-expression systems are those hosts that have lipid rafts suitable for assembly of the VLP. These include but are not limited to microorganisms such as bacteria (e.g., E. coli, B. subtilis) transformed with recombinant bacteriophage DNA, plasmid DNA or cosmid DNA expression vectors containing VLP polypeptide coding sequences; yeast (e.g., Saccharomyces, Pichia) transformed with recombinant yeast expression vectors containing VLP polypeptide coding sequences; insect cell systems infected with recombinant virus expression vectors (e.g., baculovirus) containing VLP polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing VLP polypeptide coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Mammalian cells or insect cells may be used for the expression of the VLP polypeptides where VLP assembly is driven by raft lipid association. For example, mammalian cells such as MRC-5 cells, Vero cells, PER.C6™ cells, Chinese hamster ovary cells (CHO), and HEK293 cells, in conjunction with a vector such as the major intermediate early gene promoter element from human cytomegalovirus is an effective expression system for VLP polypeptides (Foecking et al., Gene 45:101 (1986); Cockett et al., Bio/Technology 8:2 (1990)).

In an insect system, Autographa californica nuclear polyhedrosis virus (AcNPV) may be used as a vector to express foreign genes. The virus grows in Spodoptera frugiperda cells. The VLP polypeptide coding sequence(s) may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the VLP polypeptide sequence(s) of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the VLP polypeptide(s) in infected hosts. (e.g., see Logan & Shenk, Proc. Natl. Acad. Sci. USA 81:355-359 (1984)). Specific initiation signals may also be requited for efficient translation of inserted VLP polypeptide coding sequence(s). These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)). One example would be the human CMV immediate early promoter as used in adenovirus-based vector systems such as the AdEASY-XL™ system from Stratagene.

In addition, a host cell strain may be chosen which modulates the expression of the inserted sequences, or modifies and processes the gene product in the specific fashion desired. Such modifications (e.g., glycosylation) and processing (e.g., cleavage or transport to the membrane) of protein products may be important for the generation of the VLP or function of a VLP polypeptide or additional polypeptide such as an adjuvant or additional antigen. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

The host cell may be co-transfected with two expression vectors of the invention, the first vector encoding a gag polypeptide and the second vector encoding a viral membrane antigen or a lipid-raft associated polypeptide linked to an antigen. The two vectors may contain identical selectable markers which enable equal expression of each VLP polypeptide. Alternatively, a single vector may be used which encodes, and is capable of expressing, both the gag polypeptide and the lipid-raft associated polypeptide linked to an antigen

Once a VLP has been produced by a host cell, it may be purified by any method known in the art for purification of a polypeptide, for example, by chromatography (e.g., ion exchange, affinity, particularly by affinity for any affinity purification tags added to the polypeptide, and size exclusion chromatography), centrifugation, differential solubility, or by any other standard technique for the purification of proteins or other macromolecules. In addition, the VLP polypeptide can be fused to heterologous polypeptide sequences described herein or otherwise known in the art, to facilitate purification of the VLP. After purification, additional elements such as additional antigens or adjuvants may be physically linked to the VLP either through covalent linkage to the VLP polypeptides or by other non-covalent linkages mechanism. In certain embodiments where the VLP polypeptides are co-expressed in a host cell that has lipid-raft domains such as mammalian cells and insect cells, the VLPs will self assemble and release allowing purification of the VLPs by any of the above methods. Certain embodiments of VLPs include VLPs engineered from homologous virus proteins, for example VLPs constructed from M1, HA and optionally NA from influenza virus, and VLPs engineered from heterologous viruses, for example Gag protein from MLV or HIV or other retroviruses engineered to form VLPs with antigens from a different virus, for example influenza HA and NA.

Exemplary Methods of Making Gag-Based VLPs

VLPs may be readily assembled by any methods available to one of skill in the art that results in assembled VLPs including a gag polypeptide and an influenza antigen polypeptide. In certain embodiments, the polypeptides may be co-expressed in any available protein expression system, such as a cell-based system that includes raft-lipid domains in the lipids such as mammalian cell expression systems and insect cell expression systems.

Numerous examples of expression of VLPs formed using gag polypeptides have been published demonstrating the range of expression systems available for generating VLPs. Studies with several retroviruses have demonstrated that the Gag polypeptide expressed in the absence of other viral components is sufficient for VLP formation and budding at the cell surface (Wills and Craven AIDS 5, 639-654, 1991; Zhou et al., 3. Virol. 68, 2556-2569, 1994; Morikawa et al., Virology 183, 288-297, 1991; Royer et al., Virology 184, 417-422, 1991; Gheysen et al., Cell 59, 103-112, 1989; Hughes et al., Virology 193, 242-255, 1993; Yamshchikov et al. Virology 214, 50-58, 1995). Formation of VLP upon expression of the Gag precursor in insect cells using a Baculovirus vector has been demonstrated by several groups (Delchambre et al., EMBO J. 8, 2653-2660, 1989; Luo et al., Virology 179, 874-880, 1990; Royer et al., Virology 184, 417-422, 1991; Morikawa et al., Virology 183, 288-297, 1991; Zhou et al., J. Virol. 68, 2556-2569, 1994; Gheysen et al., Cell 59, 103-112, 1989; Hughes et al., Virology 193, 242-255, 1993; Yamslichikov et al., Virology 214, 50-58, 1995). These VLPs resemble immature lentivirus particles and are efficiently assembled and released by budding from the insect cell plasma membrane.

It has been reported that the amino terminal region of the Gag precursor is a targeting signal for transport to the cell surface and membrane binding which is required for virus assembly (Yu et al., J. Virol. 66, 4966-4971, 1992; an, X et al. J. Virol. 67, 6387-6394, 1993; Zhou et al., J. Virol. 68, 2556-2569, 1994; Lee and Linial J. Virol. 68, 6644-6654, 1994; Dorfman et al., J. Virol. 68, 1689-1696, 1994; Facke et al., J. Virol. 67, 4972-4980, 1993). Assembly of recombinant HIV based VLPs that contain Gag structural proteins as well as Env glycoproteins gp120 and gp41 has been reported using a vaccinia virus expression system (Haffar et al., J. Virol. 66, 4279-4287, 1992).

Exemplary Methods of Inactivation of Infectious Agents in Enveloped Virus-Based VLP Preparations

An exemplary method of inactivation is through electromagnetic radiation as electromagnetic radiation is capable of inactivating the infectious agents without substantially reducing the immunogenicity of the enveloped virus-based VLP. As all three exemplary modes of electromagnetic radiation (i.e., UV irradiation with photoreactive compounds, UV irradiation alone and gamma irradiation) have a long history of use for inactivation of pathogens in a wide variety of samples such as blood, food, vaccines, etc. there are a wide variety of commercially available apparatus for applying the inactivating electromagnetic radiation that may be used with little to no modification to practice the methods disclosed herein. Furthermore, optimizing wavelengths and dosages is routine in the art and therefore readily within the capabilities of one of ordinary skill in the art.

UV Irradiation with Photoreactive Compounds

An exemplary method of inactivation with electromagnetic radiation is a combination of ultraviolet irradiation, such as UV-A irradiation, in the presence of a photoreactive compound, such as one that will react with polynucleotides in the infectious agent.

Exemplary photoreactive compounds include: actinomycins, anthracyclinones, anthramycin, benzodipyrones, fluorenes, fluorenones, furocoumarins, isoalloxazine, mitomycin, monostral fast blue, norphillin A, phenanthridines, phenazathionium salts, phenazines, phenothiazines, phenylazides, quinolines, and thiaxanthenones. One species is furocoumarins which belong in one of two main categories. The first category is psoralens [7H-furo(3,2-g)-(1)-benzopyran-7-one, or delta-lactone of 6-hydroxy-5-benzofuranacrylic acid], which are linear and in which the two oxygen residues appended to the central aromatic moiety have a 1, 3 orientation, and further in which the furan ring moiety is linked to the 6 position of the two ring coumarin system. The second category is isopsoralens [2H-furo(2,3-h)-(1)-benzopyran-2-one, or delta-lactone of 4-hydroxy-5-benzofuranacrylic acid], which are angular and in which the two oxygen residues appended to the central aromatic moiety have a 1, 3 orientation, and further in which the furan ring moiety is linked to the 8 position of the two ring coumarin system. Psoralen derivatives may be generated by substitution of the linear furocoumarin at the 3, 4, 5, 8, 4′, or 5′ positions, while isopsoralen derivatives may be generated by substitution of the angular furocoumarin at the 3, 4, 5, 6, 4′, or 5 positions. Psoralens can intercalate between the base pain of double-stranded nucleic acids, forming covalent adducts to pyrimidine bases upon absorption of long wave ultraviolet light (UVA). See, e.g., G. D. Cimino et al., Ann. Rev. Biochem 54:1151 (1985); Hearst et al., Quart. Rev. Biophys. 17:1 (1984).

Exemplary wavelengths of UV (or in some cases visible light) radiation will depend upon the wavelength at which appropriate reactions and/or photoadducts are generated which is dependent upon the chemistry of the photoreactive chemical. By way of example, UV radiation in the wavelengths between 320 and 380 nm are most effective for many psoralens with 330 to 360 nm having maximum effectiveness. Similar UV-A wavelengths are also highly effective in conjunction with riboflavin, a photoreactive compound that can also be used coupled with visible light such as 419 nm for pathogen inactivation.

UV Irradiation Alone

In addition to UV irradiation in the presence of a photoreactive compound, infectious agents may be inactivated by UV irradiation alone. In certain embodiments, the radiation is UV-C radiation having a wavelength between about 180 and 320 nm, or between about 225 and 290 nm, or about 254 nm (i.e., spectral region with a high absorbance peak of polynucleotides and diminished protein absorption). UV-C radiation may be used because it is less detrimental to the components of the enveloped virus-based VLPs disclosed herein for both stability and inummogenicity such as the lipid bilayer forming the envelope and proteins within the envelope while retaining sufficient energy to inactivate infectious agents. However, other types of UV radiation such as, for example, UV-A and UV-B may also be used.

Gamma Irradiation

Gamma irradiation (i.e., ionizing radiation) may also be used in the practice of the methods disclosed herein to generate the compositions. In this embodiment, gamma irradiation doses of between 10 and 60 kGy are effective for pathogen inactivation. Gamma irradiation can directly inactivate infectious agents by introducing strand breaks in the polynucleotides encoding the genome of the infectious agent or indirectly by generating free radicals that attack the polynucleotides. Free radical scavengers and low temperature may be used in conjunction with gamma irradiation to inhibit radical-mediated damage to lipid and protein components of enveloped VLPs.

Exemplary Methods of Using VLPs

Formulations

An exemplary use of the enveloped-virus based VLPs described herein is as a vaccine preparation. Typically, such vaccines are prepared as injectables either as liquid solutions or suspensions; solid forms suitable for solution in, or suspension in, liquid prior to injection may also be prepared. Such preparations may also be emulsified or produced as a dry powder. The active immunogenic ingredient is often mixed with excipients which are pharmaceutically acceptable and compatible with the active ingredient. Suitable excipients are, for example, water, saline, dextrose, sucrose, glycerol, ethanol, or the like, and combinations thereof. In addition, if desired, the vaccine may contain auxiliary substances such as wetting or emulsifying agents, pH buffering agents, or adjuvants which enhance the effectiveness of the vaccines.

Vaccines may be conventionally administered parenterally, by injection, for example, either subcutaneously, intradermally, subdermally or intramuscularly. Additional formulations which are suitable for other modes of administration include suppositories and, in some cases, oral, intranasal, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, or even 1-2%. In certain embodiments, a low melting wax, such as a mixture of fatty acid glycerides or cocoa butter is first melted and the enveloped-virus based VLPs described herein are dispersed homogeneously, for example, by stirring. The molten homogeneous mixture is then poured into conveniently sized molds, allowed to cool, and to solidify.

Formulations suitable for intranasal delivery include liquids (e.g., aqueous solution for administration as an aerosol or nasal drops) and dry powders (e.g. for rapid deposition within the nasal passage). Formulations include such normally employed excipients as, for example, pharmaceutical grades of mannitol, lactose, sucrose, trehalose, xylitol, and chitosan. Mucosadhesive agents such as chitosan can be used in either liquid or powder formulations to delay mucocilliary clearance of intranasally-administered formulations. Sugars such as mannitol and sucrose can be used as stability agents in liquid formulations and as stability, bulking, or powder flow and size agents in dry powder formulations. In addition, adjuvants such as monophosphoryl lipid A (MPL) or CpG oligonucleotides can be used in both liquid and dry powder formulations as an immunostimulatory adjuvant.

Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations include such normally employed excipients as, for example, pharmaceutical grades of mannitol, lactose, starch, magnesium stearate, sodium saccharine, cellulose, magnesium carbonate, and the like. Other enveloped-virus based VLP vaccine compositions may take the form of solutions, suspensions, pills, sustained release formulations or powders and contain 10-95% of active ingredient or 25-70% or active ingredient. For oral formulations, cholera toxin is an interesting formulation partner (and also a possible conjugation partner).

The enveloped-virus based VLP vaccines when formulated for vaginal administration may be in the form of pessaries, tampons, creams, gels, pastes, foams or sprays. Any of the foregoing formulations may contain agents in addition to enveloped-virus based VLPs, such as carriers, known in the art to be appropriate.

In some embodiments, the enveloped-virus based VLP vaccine may be formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., intradermal, topical application, intravenous, intramuscular, etc.

The enveloped-virus based VLPs may be formulated into the vaccine including neutral or salt-based formulations. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, maudelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines may be administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with an exemplary range from about 0.1 μg to 2000 μg (even though higher amounts in the 1-10 mg range are contemplated), such as in the range from about 0.5 μg to 1000 μg, in the range from 1 μg to 500 μg, or in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the vaccine formulations will be sufficiently immunogenic as a vaccine by themselves, but for some of the others the immune response will be enhanced if the vaccine further comprises an adjuvant substance.

Delivery agents that improve mucoadhesion can also be used to improve delivery and immunogenicity especially for intranasal, oral or lung based delivery formulations. One such compound, chitosan, the N-deacetylated form of chitin, is used in many pharmaceutical formulations (32). It is an attractive mucoadhesive agent for intranasal vaccine delivery due to its ability to delay mucociliary clearance and allow more time for mucosal antigen uptake and processing (33, 34). In addition, it can transiently open tight junctions which may enhance transepithelial transport of antigen to the NALT. In a recent human trial, a trivalent inactivated influenza vaccine administered intranasally with chitosan but without any additional adjuvant yielded seroconversion and HI titers that were only marginally lower than those obtained following intramuscular inoculation (33).

Chitosan can also be formulated with adjuvants that function well intranasally such as the genetically detoxified E. coli heat-labile enterotoxin mutant LTK63. This adds an inmmnostimulatory effect on top of the delivery and adhesion benefits imparted by chitosan resulting in enhanced mucosal and systemic responses (35).

Finally, it should be noted that chitosan formulations can also be prepared in a dry powder format that has been shown to improve vaccine stability and result in a further delay in mucociliary clearance over liquid formulations (42). This was seen in a recent human clinical trial involving an intranasal dry powder diphtheria toxoid vaccine formulated with chitosan in which the intranasal route was as effective as the traditional intramuscular route with the added benefit of secretory IgA responses (43). The vaccine was also very well tolerated. Intranasal dry powdered vaccines for anthrax containing chitosan and MPL induce stronger responses in rabbits than intramuscular inoculation and are also protective against aerosol spore challenge (44).

Intranasal vaccines represent an exemplary formulation as they can affect the upper and lower respiratory tracts in contrast to parenterally administered vaccines which are better at affecting the lower respiratory tract. This can be beneficial for inducing tolerance to allergen-based vaccines and inducing immunity for pathogen-based vaccines.

In addition to providing protection in both the upper and lower respiratory tracts, intranasal vaccines avoid the complications of needle inoculations and provide a means of inducing both mucosal and systemic humoral and cellular responses via interaction of particulate and/or soluble antigens with nasopharyngeal-associated lymphoid tissues (NALT) (16-19). The intranasal route has been historically less effective than parenteral inoculation, but the use of enveloped-virus based VLPs, novel delivery formulations, and adjuvants are beginning to change the paradigm. Indeed, influenza vaccines containing functional hemagglutinin molecules may be especially well suited for intranasal delivery due to the abundance of sialic acid-containing receptors in the nasal mucosa resulting in the potential for enhanced HA antigen binding and reduced mucociliary clearance.

With respect to influenza, protective immune responses including heterosubtypic protection have been reported following intranasal vaccine delivery in experiments where parallel parenteral administrations were less immunogenic and did not induce heterosubtypic protection (20-22). Moreover, inactivated influenza has been shown to be an effective adjuvant for systemic and mucosal humoral and cellular responses when admixed with a simian immunodeficiency virus (SIV) VLP vaccine administered intranasally (23). This adjuvant effect was attributed to the ability of inactivated influenza virions to aggregate with the VLPs and lead to enhanced binding to mucosal surfaces. A similar adjuvant effect was also seen when influenza HA was directly incorporated into SIV VLPs which led to enhanced binding to and activation of dendritic cells (DC) (24, 25).

Adjuvants

Various methods of achieving adjuvant effect for vaccines are known and may be used in conjunction with the enveloped-virus based VLPs disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

In some embodiments, a VLP vaccine comprises the enveloped-virus based VLP in admixture with at least one adjuvant, at a weight-based ratio of from about 10:1 to about 10¹⁰:1 VLP:adjuvant, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10³:1, from about 10⁸:1 to about 10⁹:1, or from about 10⁹:1 to about 10¹⁰:1 VLP:adjuvant. One of skill in the art can readily determine the appropriate ratio through information regarding the adjuvant and routine experimentation to determine optimal ratios. One of skill in the art can readily determine the appropriate ratio through information regarding the adjuvant and routine experimentation to determine optimal ratios. Admixtures of VLPs and adjuvants as disclosed herein may include any form of combination available to one of skill in the art including, without limitation, mixture of separate VLPs and adjuvants in the same solution, covalently linked VLPs and adjuvants, ionically linked VLPs and adjuvants, hydrophobically linked VLPs and adjuvants (including being embedded partially or fully in the VLP membrane), hydrophilically linked VLPs and adjuvants, and any combination of the foregoing.

Exemplary adjuvants may include, but are not limited to, toll-like receptor (TLR) agonists, monophosphoryl lipid A (MPL), synthetic lipid A, lipid A mimetics or analogs, aluminum salts, cytokines, saponins, muramyl dipeptide (MDP) derivatives, CpG oligos, lipopolysaccharide (LPS) of gram-negative bacteria, polyphosphazenes, emulsions, oil in water emulsions, virosomes, cochleates, poly(lactide-co-glycolides) (PLG) microparticles, poloxamer particles, microparticles, and liposomes. Preferably, the adjuvants are not bacterially-derived exotoxins. Preferred adjuvants are those which stimulate a Th1 type response such as 3DMPL, CpG oligonucleotides, or QS21.

Monophosphoryl Lipid A (MPL), a non-toxic derivative of lipid A from Salmonella, is a potent TLR-4 agonist that has been developed as a vaccine adjuvant (Evans et al. 2003). In pre-clinical murine studies intranasal MPL has been shown to enhance secretory, as well as systemic, humoral responses (Baldridge et al. 2000; Yang et al. 2002). It has also been proven to be safe and effective as a vaccine adjuvant in clinical studies of greater than 120,000 patients (Baldrick et al., 2002; 2004). MPL stimulates the induction of innate immunity through the TLR-4 receptor and is thus capable of eliciting nonspecific immune responses against a wide range of infectious pathogens, including both gram negative and gram positive bacteria, viruses, and parasites (Baldrick et al. 2004; Persing et al. 2002). Inclusion of MPL in intranasal formulations should provide rapid induction of innate responses, eliciting nonspecific immune responses from viral challenge while enhancing the specific responses generated by the antigenic components of the vaccine.

Accordingly, in one embodiment, the present invention provides a composition comprising monophosphoryl lipid A (MPL®) or 3 De-O-acylated monophosphoryl lipid A (3D-MPL®) as an enhancer of adaptive and innate immunity. Chemically 3D-MPL® is a mixture of 3 De-O-acylated monophosphoryl lipid A with 4, 5 or 6 acylated chains. An exemplary form of 3 De-O-acylated monophosphoryl lipid A is disclosed in European Patent 0 689 454 B1 (SmithKline Beecham Biologicals SA. In another embodiment, the present invention provides a composition comprising synthetic lipid A, lipid A mimetics or analogs, such as BioMira's PET Lipid A, or synthetic derivatives designed to function like TLR-4 agonists.

Exemplary adjuvants are polypeptide adjuvants that may be readily added to the enveloped-virus based VLPs described herein by co-expression with the VLP polypeptides or fusion with the VLP polypeptides to produce chimeric polypeptides. Bacterial flagellin, the major protein constituent of flagella, is an adjuvant which has received increasing attention as an adjuvant protein because of its recognition by the innate immune system by the toll-like receptor TLR5 (65). Flagellin signaling through TLR5 has effects on both innate and adaptive immune functions by inducing DC maturation and migration as well as activation of macrophages, neutrophils, and intestinal epithelial cells resulting in production of proinflammatory mediators (66-72).

TLR5 recognizes a conserved structure within flagellin monomers that is unique to this protein and is required for flagellar function, precluding its mutation in response to immunological pressure (73). The receptor is sensitive to a 100 fM concentration but does not recognize intact filaments. Flagellar disassembly into monomers is required for binding and stimulation.

As an adjuvant, flagellin has potent activity for induction of protective responses for heterologous antigens administered either parenterally or intranasally (66, 74-77) and adjuvant effects for DNA vaccines have also been reported (78). A Th2 bias is observed when flagellin is employed which would be appropriate for a respiratory virus such as influenza but no evidence for IgE induction in mice or monkeys has been observed. In addition, no local or systemic inflammatory responses have been reported following intranasal or systemic administration in monkeys (74). The Th2 character of responses elicited following use of flagellin is somewhat surprising since flagellin signals through TLR5 in a MyD88-dependent manner and all other MyD88-dependent signals through TLRs have been shown to result in a Th1 bias (67, 79). Importantly, pre-existing antibodies to flagellin have no appreciable effect on adjuvant efficacy (74) making it attractive as a multi-use adjuvant.

A common theme in many recent intranasal vaccine trials is the use of adjuvants and/or delivery systems to improve vaccine efficacy. In one such study an influenza H3 vaccine containing a genetically detoxified E. coli heat-labile enterotoxin adjuvant (LT R192G) resulted in heterosubtypic protection against H5 challenge but only following intranasal delivery. Protection was based on the induction of cross neutralizing antibodies and demonstrated important implications for the intranasal route in development of new influenza vaccines (22).

Cytokines, colony-stimulating factors (e.g., GM-CSF, CSF, and the like); tumor necrosis factor; interleukin-2, -7, -12, and other like growth factors, may also be used as adjuvants as they may be readily included in the enveloped-virus based VLP vaccine by admixing or fusion with the VLP polypeptides.

In some embodiments, the enveloped-virus based VLP vaccine compositions disclosed herein may include other adjuvants that act through a Toll-like receptor such as a nucleic acid TLR9 ligand comprising a CpG oligonucleotide; an imidazoquinoline TLR7 ligand; a substituted guanine TLR7/8 ligand; other TLR7 ligands such as Loxoribine, 7-deazadeoxyguanosine, 7-thia-8-oxodeoxyguanosine, double stranded poly (I:C), poly-inosinic acid, Imiquimod (R-837), and Resiquimod (R-848); or a TLR4 agonist such as MPL® or synthetic derivatives.

Certain adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminum adjuvants; DNA adjuvants; MPL; and an encapsulating adjuvant.

Additional examples of adjuvants include agents such as aluminum salts such as hydroxide or phosphate (alum), commonly used as 0.05 to 0.1 percent solution in buffered saline (see, e.g., Nicklas (1992) Res. Immunol. 143:489-493), admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and WA may also be used.

DDA (dimethyldioctadecylammonium bromide) is an interesting candidate for an adjuvant, but also Freund's complete and incomplete adjuvants as well as quillaja saponins such as QuilA and QS21 are interesting. Further possibilities include poly[di(earboxylatophenoxy)phosphazene (PCPP) derivatives of lipopolysaccharides such as monophosphoryl lipid A (MPL®), muramyl dipeptide (MDP) and threonyl muramyl dipeptide (tMDP). The lipopolysaccharide based adjuvants may be used to produce a predominantly Th1-type response including, for example, a combination of monophosphoryl lipid A, such as 3-de-O-acylated monophosphoryl lipid A, together with an aluminum salt. MPL® adjuvants are available from GlaxoSmithKline (see, for example, U.S. Pat. Nos. 4,436,727; 4,877,611; 4,866,034 and 4,912,094, each of which is incorporated by reference in their entirety with particular reference to their lipopolysaccharides related teachings).

Liposome formulations are also known to confer adjuvant effects, and therefore liposome adjuvants may be used in conjunction with the enveloped-virus based VLPs.

Immunostimulating complex matrix type (ISCOM® matrix) adjuvants may also be used with the enveloped-virus based VLP vaccines, especially since it has been shown that this type of adjuvants are capable of up-regulating MHC Class II expression by APCs. An ISCOM matrix consists of (optionally fractionated) saponins (triterpenoids) from Quillaja saponaria, cholesterol, and phospholipid. When admixed with the immunogenic protein such as in the VPLs, the resulting particulate formulation is what is known as an ISCOM particle where the saponin may constitute 60-70% w/w, the cholesterol and phospholipid 10-15% w/w % and the protein 10-15% w/w. Details relating to composition and use of immunostimulating complexes can for example be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

The saponins, whether or not in the form of ISCOMs, that may be used in the adjuvant combinations with the enveloped-virus based VLP vaccines disclosed herein include those derived from the bark of Quillaja Saponaria Molina, termed Quil A, and fractions thereof, described in U.S. Pat. No. 5,057,540 (which is incorporated by reference herein in its entirety with particular reference to the fractions of Quil A and methods of isolation and use thereof) and “Saponins as vaccine adjuvants”, Kensil, C. R., Crit Rev Ther Drug Carrier Syst, 1996, 12 (1-2):1-55; and EP 0 362 279 B1. Exemplary fractions of Quil A are QS21, QS7, and QS17.

β-Escin is another hemolytic saponins for use in the adjuvant compositions of the enveloped-virus based VLP vaccines described herein. Escin is described in the Merck index (12th ed: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 August; 44(8):1454-1464)). β-escin is also known as aescin.

Another hemolytic saponin for use with the enveloped-virus based VLP vaccines is Digitonin. Digitonin is described in the Merck index (12^{th} Edition, entry 3204) as a saponin, being derived from the seeds of Digitalis purpurea and purified according to the procedure described Gisvold et al., J. Am. Pharm. Assoc., 1934, 23, 664; and Ruhenstroth-Bauer, Physiol. Chem., 1955, 301, 621. Its use is described as being a clinical reagent for cholesterol determination.

Another interesting possibility of achieving adjuvant effect is to employ the technique described in Gosselin et al., 1992 (which is hereby incorporated by reference herein). In brief; the presentation of a relevant antigen such as the VLP polypeptides or additional antigens described herein can be enhanced by conjugating the antigen to antibodies (or antigen binding antibody fragments) against the F_{c} receptors on monocytes/macrophages. Especially conjugates between antigen and anti-F_{c}RI have been demonstrated to enhance immunogenicity for the purposes of vaccination. The antibody may be conjugated to the enveloped-virus based VLP after generation or as a part of the generation including by expressing as a fusion to any one of the VLP polypeptides.

Other possibilities involve the use of the targeting and immune modulating substances (i.e. cytokines). In addition, synthetic inducers of cytokines such as poly I:C may also be used.

Suitable mycobacterial derivatives may be selected from the group consisting of muramyl dipeptide, complete Freund's adjuvant, and a diester of trehalose such as TDM and TDE.

Examples of suitable immune targeting adjuvants include CD40 ligand and CD40 antibodies or specifically binding fragments thereof (cf. the discussion above), mannose, a Fab fragment, and CTLA-4.

Examples of suitable polymer adjuvants include a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12-15, 1998, Seascape Resort, Aptos, Calif.”

Oligonucleotides may be used as adjuvants in conjunction with the enveloped-virus based VLP vaccines and may contain two or more dinucleotide CpG motifs separated by at least three or at least six or more nucleotides. CpG-containing oligonucleotides (in which the CpG dinucleotide is =methylated) induce a predominantly Th1 response. Such oligonucleotides are well known and are described, for example, in WO 96/02555, WO 99/33488 and U.S. Pat. Nos. 6,008,200 and 5,856,462, each of which is hereby incorporated by reference in their entirety with particular reference to methods of making and using CpG oligonucleotides as adjuvants.

Such oligonucleotide adjuvants may be deoxynucleotides. In certain embodiments, the nucleotide backbone in the oligonucleotide is phosphorodithioate or a phosphorothioate bond, although phosphodiester and other nucleotide backbones such as PNA may be used with the enveloped-virus based VLP vaccines including oligonucleotides with mixed backbone linkages. Methods for producing phosphorothioate oligonucleotides or phosphorodithioate are described in U.S. Pat. No. 5,666,153, U.S. Pat. No. 5,278,302 and W095/26204, each of which is hereby incorporated by reference in their entirety with particular reference to the phosphorothioate and phosphorodithioate teachings.

Exemplary oligonucleotides have the following sequences. The sequences may contain phosphorothioate modified nucleotide backbones.

(SEQ ID NO: 1)
OLIGO 1: TCC ATG ACG TTC CTG ACG TT (CpG 1826)
(SEQ ID NO: 2)
OLIGO 2: TCT CCC AGC GTG CGC CAT (CpG 1758)
(SEQ ID NO: 3)
OLIGO 3: ACC GAT GAC GTC GCC GGT GAC GGC ACC ACG
(SEQ ID NO: 4)
OLIGO 4: TCG TCG TTT TGT CGT TTT GTC GTT (CpG
2006)
(SEQ ID NO: 5)
OLIGO 5: TCC ATG ACG TTC CTG ATG CT (CpG 1668)

Alternative CpG oligonucleotides include the above sequences with inconsequential deletions or additions thereto. The CpG oligonucleotides as adjuvants may be synthesized by any method known in the art (e.g., EP 468520). For example, such oligonucleotides may be synthesized utilizing an automated synthesizer. Such oligonucleotide adjuvants may be between 10-50 bases in length. Another adjuvant system involves the combination of a CpG-containing oligonucleotide and a saponin derivative particularly the combination of CpG and QS21 is disclosed in WO 00/09159.

Many single or multiphase emulsion systems have been described. One of skill in the art may readily adapt such emulsion systems for use with enveloped-virus based VLPs so that the emulsion does not disrupt the enveloped-virus based VLP's structure. Oil in water emulsion adjuvants per se have been suggested to be useful as adjuvant compositions (EPO 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use with the enveloped-virus based VLP vaccines described herein may be natural or synthetic, and may be mineral or organic. Examples of mineral and organic oils will be readily apparent to the man skilled in the art.

In order for any oil in water composition to be suitable for human administration, the oil phase of the emulsion system may include a metabolizable oil. The meaning of the term metabolizable oil is well known in the art. Metabolizable can be defined as “being capable of being transformed by metabolism” (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils may also be used with the enveloped-virus based VLP vaccines and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and may be used with the enveloped-virus based VLP vaccines disclosed herein. Squalene is a metabolizable oil virtue of the fact that it is an intermediate in the biosynthesis of cholesterol (Merck index, 10th Edition, entry no. 8619).

Exemplary oil emulsions are oil in water emulsions, and in particular squalene in water emulsions.

In addition, the oil emulsion adjuvants for use in the enveloped-virus based VLP vaccines may comprise an antioxidant, such as the oil α-tocopherol (vitamin E, EP 0 382 271 B1).

WO 95/17210 and WO 99/11241 disclose emulsion adjuvants based on squalene, α-tocopherol, and TWEEN 80™ optionally formulated with the immunostimulants QS21 and/or 3D-MPL. WO 99/12565 discloses an improvement to these squalene emulsions with the addition of a sterol into the oil phase. Additionally, a triglyceride, such as tricaprylin (C27H50O6), may be added to the oil phase in order to stabilize the emulsion (WO 98/56414).

The size of the oil droplets found within the stable oil in water emulsion may be less than 1 micron, may be in the range of substantially 30-600 nm, substantially around 30-500 nm in diameter, substantially 150-500 nm in diameter, or about 150 nm in diameter as measured by photon correlation spectroscopy. In this regard, 80% of the oil droplets by number may be within these ranges, more than 90% or more than 95% of the oil droplets by number are within the defined size ranges. The amounts of the components present in the oil emulsions are conventionally in the range of from 2 to 10% oil, such as squalene; and when present, from 2 to 10% alpha tocopherol; and from 0.3 to 3% surfactant, such as polyoxyethylene sorbitan monooleate. The ratio of oil:alpha tocopherol may be equal or less than 1 as this provides a more stable emulsion. SPAN 85™ may also be present at a level of about 1%. In some cases it may be advantageous that the enveloped-virus based VLP vaccines disclosed herein will further contain a stabilizer.

The method of producing oil in water emulsions is well known to the man skilled in the art. Commonly, the method comprises the mixing the oil phase with a surfactant such as a PBS/TWEEN80® solution, followed by homogenization using a homogenizer, it would be clear to a man skilled in the art that a method comprising passing the mixture twice through a syringe needle would be suitable for homogenizing small volumes of liquid. Equally, the emulsification process in microfluidizer (M110S microfluidics machine, maximum of 50 passes, for a period of 2 minutes at maximum pressure input of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

The enveloped-virus based VLP vaccine preparations disclosed herein may be used to protect or treat a mammal or bird susceptible to, or suffering from viral influenza, by means of administering said vaccine by intranasal, intramuscular, intraperitoneal, intradermal, transdermal, intravenous, or subcutaneous administration. Methods of systemic administration of the vaccine preparations may include conventional syringes and needles, or devices designed for ballistic delivery of solid vaccines (WO 99/27961), or needleless pressure liquid jet device (U.S. Pat. No. 4,596,556; U.S. Pat. No. 5,993,412), or transdermal patches (WO 97/48440; WO 98/28037). The enveloped-virus based VLP vaccines may also be applied to the skin (transdermal or transcutaneous delivery WO 98/20734; WO 98/28037). The enveloped-virus based VLP vaccines disclosed herein therefore may include a delivery device for systemic administration, pre-filled with the enveloped-virus based VLP vaccine or adjuvant compositions. Accordingly there is provided a method for inducing an immune response in an individual such as a mammal or bird, comprising the administration of a vaccine comprising any of the enveloped-virus based VLP compositions described herein and optionally including an adjuvant and/or a carrier, to the individual, wherein the vaccine is administered via the parenteral or systemic route.

The enveloped-virus based VLP vaccine preparations disclosed herein may be used to protect or treat a mammal or bird susceptible to, or suffering from viral influenza, by means of administering said vaccine via a mucosal route, such as the oral/alimentary or nasal route. Alternative mucosal routes are intravaginal and intra-rectal. Exemplary mucosal route of administration may be via the nasal route, termed intranasal vaccination. Methods of intranasal vaccination are well known in the art, including the administration of a droplet, spray, or dry powdered form of the vaccine into the nasopharynx of the individual to be immunized. Nebulized or aerosolized vaccine formulations are exemplary forms of the enveloped-virus based VLP vaccines disclosed herein. Enteric formulations such as gastro resistant capsules and granules for oral administration, suppositories for rectal or vaginal administration are also formulations of the enveloped-virus based VLP vaccines disclosed herein.

The exemplary enveloped-virus based VLP vaccine compositions disclosed herein, represent a class of mucosal vaccines suitable for application in humans to replace systemic vaccination by mucosal vaccination.

The enveloped-virus based VLP vaccines may also be administered via the oral route. In such cases the pharmaceutically acceptable excipient may also include alkaline buffers, or enteric capsules or microgranules. The enveloped-virus based VLP vaccines may also be administered by the vaginal route. In such cases, the pharmaceutically acceptable excipients may also include emulsifiers, polymers such as CARBOPOL®, and other known stabilizers of vaginal creams and suppositories. The enveloped-virus based VLP vaccines may also be administered by the rectal route. In such cases the excipients may also include waxes and polymers known in the art for forming rectal suppositories.

Alternatively the enveloped-virus based VLP vaccines formulations may be combined with vaccines vehicles composed of chitosan (as described above) or other polycationic polymers, polylactide and polylactide-coglycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM.

Additional illustrative adjuvants for use in the pharmaceutical and vaccine compositions using enveloped-virus based VLPs as described herein include SAF (Chiron, Calif., United States). MF-59 (Chiron, see, e.g., Granoff et al. (1997) Infect Immun. 65 (5):1710-1715), the SBAS series of adjuvants (e.g., SB-AS2 (SmithKline Beecham adjuvant system #2; an oil-in-water emulsion containing MPL and QS21); SBAS-4 (SmithKline Beecham adjuvant system #4; contains alum and MPL), available from SmithKline Beecham, Rixensart, Belgium), Detox (Enhanzyn®) (GlaxoSmithKline), RC-512, RC-522, RC-527, RC-529, RC-544, and RC-560 (GlaxoSmithKline) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Other examples of adjuvants include, but are not limited to, Hunter's TiterMax® adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 143:553-557); alum (e.g., aluminum hydroxide, aluminum phosphate) emulsion based formulations including mineral oil, non-mineral oil, water-in-oil or oil-in-water emulsions, such as the Seppic ISA series of Montamide adjuvants (e.g., ISA-51, ISA-57, ISA-720, ISA-151, etc.; Seppic, Paris, France); and PROVAX® (IDEC Pharmaceuticals); OM-174 (a glucosamine disaccharide related to lipid A); Leishmania elongation factor; non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

Other adjuvants include adjuvant molecules of the general formula.

HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the enveloped-virus based VLP vaccine formulations described herein include a polyoxyethylene ether of general formula (I), wherein n is between 1 and 50, 4-24, or 9; the R component is C₁₋₅₀, C₄-C₂₀ alkyl or C₁₂ alkyl, and A is a bond. The concentration of the polyoxyethylene ethers should be in the range 0.1-20%, in the range 0.1-10%, or in the range 0.1-1%. Exemplary polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12th edition: entry 7717). These adjuvant molecules are described in WO 99/52549.

The polyoxyethylene ether according to the general formula (I) above may, if desired, be combined with another adjuvant. For example, an adjuvant combination may include the CpG as described above.

Further examples of suitable pharmaceutically acceptable excipients for use with the enveloped-virus based VLP vaccines disclosed herein include water, phosphate buffered saline, isotonic buffer solutions.

Additional Influenza Antigens

The stabilized VLPs disclosed herein may include additional antigens from influenza to increase the immunogenicity with respect to particular strains of influenza and/or across multiple strains of influenza.

An exemplary additional influenza antigen is the M2 polypeptide (also called BM2 in influenza B). The M2 polypeptide of influenza virus is a small 97 amino acid class III integral membrane protein encoded by RNA segment 7 (matrix segment) following a splicing event (80, 81). Very little M2 exists on virus particles but it can be found more abundantly on infected cells. M2 serves as a proton-selective ion channel that is necessary for viral entry (82, 83). It is minimally immunogenic during infection or conventional vaccination, explaining its conservation, but when presented in an alternative format it is more immunogenic and protective (84-86). This is consistent with observations that passive transfer of an M2 monoclonal antibody in vivo accelerates viral clearance and results in protection (87). When the M2 external domain epitope is linked to HBV core particles as a fusion protein it is protective in mice via both parenteral and intranasal inoculation and is most immunogenic when three tandem copies are fused to the N-terminus of the core protein (88-90). This is consistent with other carrier-hapten data showing that increased epitope density increases immunogenicity (91).

For intranasal delivery of an M2 vaccine an adjuvant may be required to achieve good protection and good results have been achieved with LTR192G (88, 90) and CTA 1-DD (89). The peptide can also be chemically conjugated to a carrier such as KLH, or the outer membrane protein complex of N. meningitides, or human papilloma virus VLPs and is protective as a vaccine in mice and other animals (92, 93).

Insofar as the M2 protein is highly conserved it is not completely without sequence divergence. The M2 ectodomain epitopes of common strains A/PR/8/34 (H1N1) and A/Aichi/68 (H3N2) were shown to be immunologically cross reactive with all other modern sequenced human strains except for A/Hong Kong/156/97 (H5N1) (92). Examination of influenza database sequences also shows similar divergence in the M2 sequence of other more recent pathogenic H5N1 human isolates such as A/Vietnam/1203/04. This finding demonstrates that a successful H5-specific pandemic vaccine incorporating M2 epitopes will need to reflect the M2 sequences that are unique to the pathogenic avian strains rather than M2 sequences currently circulating in human H1 and H3 isolates.

Additional proteins from influenza virus (other than HA, NA and M2) may be included in the VLP vaccine either by co-expression or via linkage of all or part of the additional antigen to the gag or HA polypeptides. These additional antigens include PB2, PB1, PA, nucleoprotein, matrix (M1), BM2, NS, NS1, and NS2. For Influenza A, examples include: PB2, PB1, PA, nucleoprotein, Matrix (M1), M2, NS1, and NS2. For Influenza B, examples include: HA, NA, NP, M, PB1, PB2, PA, NS and BM2. These latter antigens are not generally targets of neutralizing antibody responses but may contain important epitopes recognized by T cells. T cell responses induced by a VLP vaccine to such epitopes may prove beneficial in boosting protective immunity.

### EXAMPLES

### Example 1

### Production of an Influenza Antigen Enveloped Virus-Based Virus-Like Particle

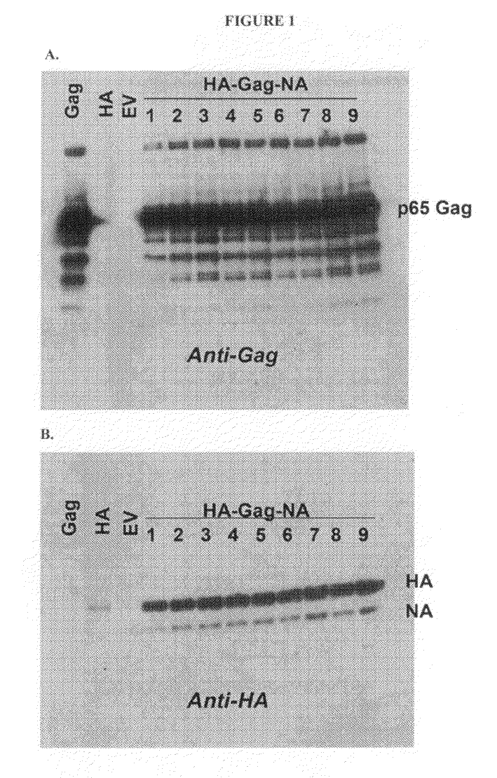

The MLV gag coding sequence was obtained by PCR from plasmid pAMS (ATCC) containing the entire Moloney murine leukemia virus amphotropic proviral sequence. The gag coding sequence was inserted into pFastBac1 (Invitrogen) behind the polyhedron promoter and the resulting plasmid was transformed into DH10Bac competent cells for recombination into the baculovirus genome. High molecular weight bacmid DNA was then purified and transfected into Sf9 cells for generation of a gag-expressing recombinant baculovirus. Two other recombinant baculoviruses encoding the hemagglutinin and neuraminidase, respectively, of A/PR/8/34 (H1N1) were produced in a similar fashion after RT-PCR cloning of the HA and NA coding sequences from virus RNA. Finally, a single baculovirus vector encoding all three products (HA-gag-NA) was produced by combining the HA, gag, and NA expression units (polyhedron promoter—coding sequence—polyA site) from individual pFastBac1 plasmids into a single pFastBac1 vector. For initial analysis, recombinant baculoviruses encoding gag or HA or gag-HA-NA were infected into Sf9 cells in 6 well plates at an MOI of >1. Three days following infection, medium supernatants were clarified of debris then pelleted at 100,000×g through a 20% sucrose cushion. Pellets were analyzed by Western blot analysis using gag and H1N1-specific antisera (See FIGS. 1A and B).

The left three lanes on each blot in FIGS. 1A and B, respectively, show the results of infecting Sf9 cells with separate gag or HA or control (EV=empty vector) baculoviruses prior to harvesting the medium. As expected, infection with a gag-only baculovirus results in significant amounts of gag antigen in the high molecular weight medium fraction due to VLP budding (FIG. 1A, lane “Gag”). In contrast, infection with an HA only baculovirus, results in little HA released into the medium on its own (FIG. 1B; lane “HA”). However, infection of Sf9 cells with a HA-gag-NA triple vector results in significant amounts of both gag and HA appearing in the 100,000×g fraction (lanes 1-9, FIGS. 1A and B) showing that gag expression can pull HA out of the cell.

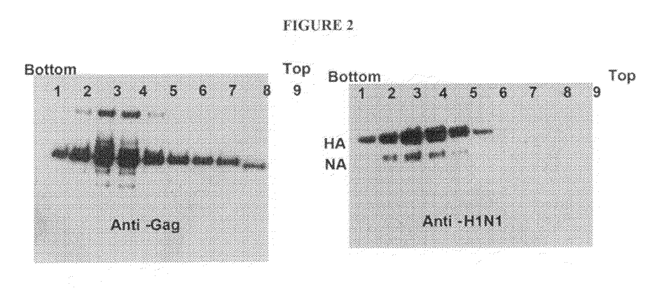

The FIGS. 2A and B show the results of recentrifugation of pelleted HA-gag-NA VLPs on a 20-60% sucrose step gradient followed by Western blot analysis of individual gradient fractions. Both gag and HA peak in the same fraction demonstrating coincident banding at a density of approximately 1.16 g/ml which indicates that the gag and HA were in VLPs.

### Example 2

### Biophysical Characterization of VLP Stability—pH and Temperature

Concentrations are reported in molarity or as percent weight-by-volume. 6-dodecanoyl-2-dimethylaminonaphthalene (laurdan) and 8-anilino-1-naphthalene sulphonate (ANS) were purchased from Molecular Probes (Eugene, Oreg.). A 1.2 mM stock solution of laurdan and a 10 mM solution of ANS were prepared by dissolution in dimethylsulfoxide (DMSO, Fisher Chemical).

Preparation of VLPs for Characterization

VLPs were produced in cultured Sf9 cells infected with a “triple gene” recombinant baculovirus as provided in Example 1. VLPs were prepared for characterization by dialysis into CP buffer at each unit pH from 4 to 8. Buffer ionic strength was maintained at 0.1 using NaCl. Material recovered from the dialysis cassettes (10,000 MWCO, Pierce, Rockford, Ill.) was concentrated at 4° C. with an Amicon® Ultra ultracentrifugation device (10,000 MWCO. Millipore, Billerica, Mass.) at 3,150×g. The protein concentration of the retentate was estimated by a BCA (bicinchoninic acid) colorimetric technique (Pierce, Rockford, Ill.). Unless otherwise noted, triplicate samples were prepared at a final protein concentration of 90 μg/mL by diluting the retentate with 20 mM CP buffer of the appropriate pH.

Trypsin Treatment of Surface Hemagglutinin

Trypsin (Sigma, final concentration 5 μg/mL) was added to VLP stock solutions (0.26 mg/mL total protein in 30% sucrose/Tris-buffered saline, pH 7.4) and incubated for 5 min in a 2-8° C. cold room. After incubation, a three-fold molar excess of trypsin inhibitor from soybean (Fluka) was added and the resulting solution passed through a 0.45-μm syringe filter (Millipore). Samples were then dialyzed into the appropriate CP buffer and concentrated as described above, but using 100,000 MWCO dialysis tubing (Spectrum Laboratories, Rancho Dominguez, Calif.). Cleavage of HA, as well as non-cleavage of MLV gag, was confirmed by western blot analysis.

Dynamic Light Scattering

Dynamic light scattering (DLS) was used to measure changes in the mean effective diameter of VLPs as a function of increasing temperature. Measurements were taken with a Brookhaven Instrument Corporation system (Holtzille, N.Y.). Incident light at 532 nm was generated by a 125 mW diode-pumped laser. Scattered light was collected at 90° to the incident beam, and a digital autocorrelator (BI-9000AT) was used to create the autocorrelation function. Five measurements were taken every 2.5° C. over the range of 10-85° C. Cumulant analysis was used to extract particle diffusion coefficients from the correlation function and convert them to particle diameters by means of the Stokes-Einstein equation. It should be noted that the effective diameter calculated by this method is accurate for particles of diameter <1 μm—the values obtained from measurements of larger particles should be used for qualitative comparison only. In addition to particle size, the second cumulant of the distribution of particle diffusion coefficients was also extracted from the correlation function as a measure of sample polydispersity.

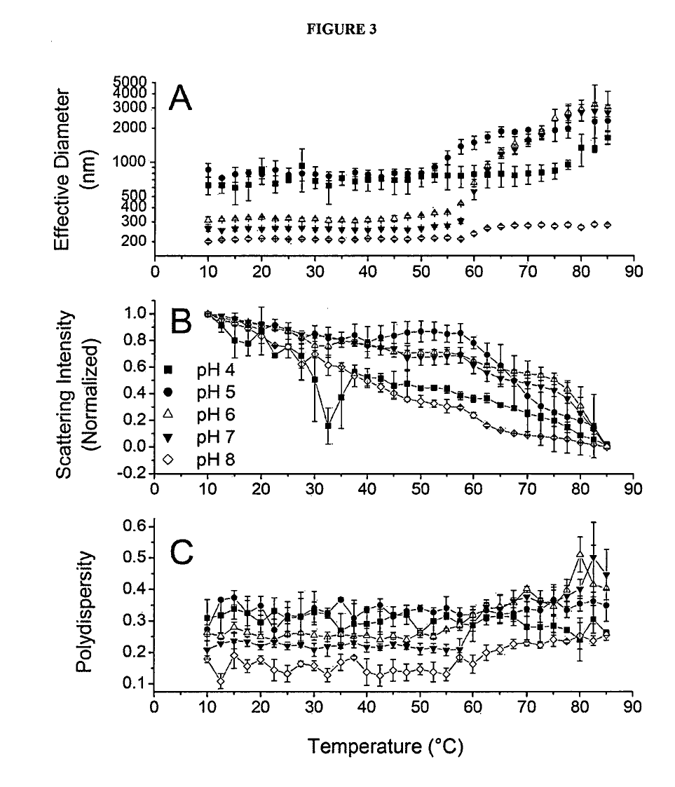

DLS measurements of VLP suspensions made using the protocol of Example 1 showed evidence of both pH- and temperature-induced changes in particle size (FIG. 3(A)). At low temperatures, the particle size at pH 4 or 5 was 2-3 times greater than at pH 6-8, indicating that significant aggregation and/or swelling was induced by acidic pH. Samples at pH 4 do not show a temperature-induced change in particle size until about 75° C., after which a gradual increase in effective diameter was observed. On the other hand, a sharp increase in particle size at pH 5 was seen at about 50° C., with another possible increase at about 75-80° C. Samples at each pH from 6-8 were stable to increasing temperature up until about 58° C., above which samples at pH 6 and 7 showed a marked increase in particle size. Samples at pH 8 also showed evidence of an increase in size at about 60° C. The size increase in the latter case was relatively small and may have been due to swelling of VLPs rather than aggregation. In general, the polydispersity of the VLPs (FIG. 3(C)) was seen to increase with increasing acidity. The polydispersity of samples at pH 4 and 5 remained nearly constant across the temperature range, while samples at pH 6 and above show an increase in polydispersity near 60° C., consistent with the changes seen in the size data.

The intensity of scattered light was also recorded during the DLS measurements (FIG. 3(B)). Normalized values are reported because instrument settings are optimized for each sample, precluding meaningful direct sample-to-sample comparisons. In general, an increase in particle size or particle refractive index (relative to the solvent) will result in an increase in scattered light intensity. It should be noted, however, that a reduction in refractive index as a result of decreased particle density (e.g. due to swelling) would manifest itself as a lower scattering intensity. This is the best explanation of the data for samples at pH 8, where a smooth and gradual reduction of scattered light intensity is seen over the majority of the temperature ramp. There is a slight disruption of the curvilinear decline around 60° C. that corresponds to the increase in effective diameter seen for these samples (FIG. 3(A)). Plots of light scattered by samples at pH 5 also show evidence of structural alterations occurring around 60° C., manifested by a sharp decrease in scattered light intensity. The plots corresponding to pH 6 and 7 are similar, with the decrease in scattered light occurring at higher temperatures (−75° C.). These decreases could be due to settling of precipitated material out of the incident light beam, consistent with the interpretation of the size data given above. The trace for pH 4 shows only a gradual decrease in intensity, with no sharp changes that correlate with measured changes in particle diameter (an exception is the transient reduction in scattering intensity from 30-35° C., but see results of laurdan fluorescence below).

Circular Dichroism Spectroscopy

Circular dichroism spectroscopy (CD) measurements were made with a Jasco J-810 spectrophotometer, using a sensitivity setting of 100 mdeg, a response time of 2 sec, and a band width of 1 nm. Composite (3-5 accumulations) spectra of VLPs were obtained at a scan rate of 20 nm/sec and a data pitch of 0.5 nm/sec. Variable temperature experiments monitoring the CD signal at 227 nm were conducted to detect changes in total VLP protein secondary structure as a function of temperature. Measurements were taken every 0.5° C. over the range of 10-90° C., with a temperature ramp rate of 15° C./h and a delay time of 2 sec. Midpoint transition temperature (Tₘ) values were determined by mathematically fitting the temperature dependent data to a sigmoidal function using the Origin® data analysis software. Both the spectra and the heating traces reflect additive contributions from the three different proteins, although they are presumably dominated by contributions from the most abundant of these proteins. The MLV gag protein has been shown to be 3-4 times more abundant than the HA protein in these VLPs, while the abundance of HA is perhaps an order of magnitude greater than that of NA.

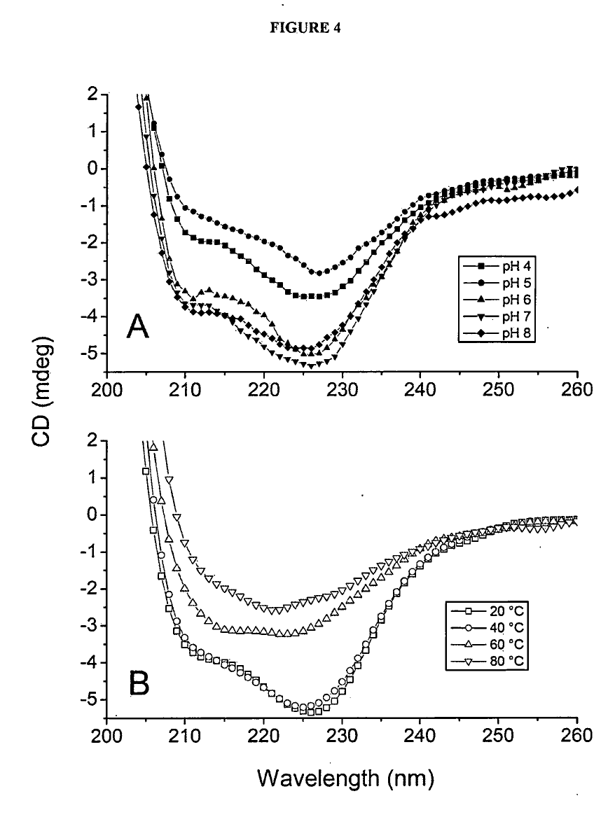

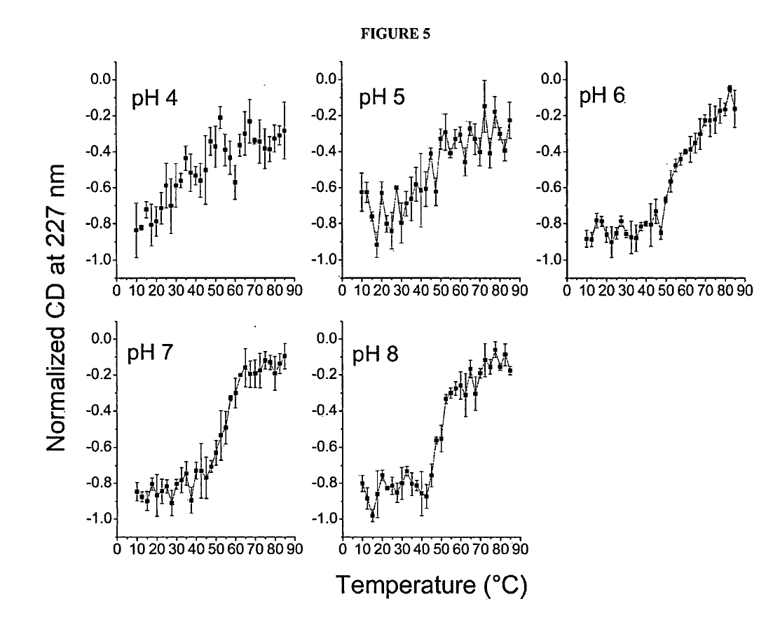

From pH 4 to 8, the CD spectra of influenza VLPs displayed minima near 210 and 227 nm, suggesting the presence of significant helical character across the pH range of interest (FIG. 4(A)). The loss of signal with increasing temperature (FIG. 4(B)) was indicative of a temperature-dependent loss of secondary structure. To further investigate this effect, the signal at. 227 nm was monitored as a function of temperature (FIG. 5). Observed sharp changes in the CD signal were consistent with temperature-dependent protein structural transitions. Samples at pH 6 show the highest Tin at around 55° C. Samples of increasing acidity have markedly reduced Tₘ values of 38 (pH 4) and 47° C. (pH 5), while samples at pH 7 or 8 are very similar with Tₘ values of 53 and 51° C., respectively. The shapes of the melting curves suggest multiple components, presumably reflecting the heterogeneous nature of the system.

Fluorescence Spectroscopy

Unless otherwise noted, fluorescence emission spectra were collected every 2.5° C. over the range 10-85° C. using a Photon Technology International fluorometer (Birmingham, N.J.). The temperature was increased at a rate of 15° C./h, with a step size of 1 nm and an integration time of 1 sec used for all measurements. Static light scattering was also monitored during fluorescence experiments through the use of a second detector (oriented 180° from the fluorescence detector). Using the Origin® software package, emission peak positions were determined by derivative analysis and Tₘ values were determined by mathematically fitting the temperature dependent data to a sigmoidal function.

The intrinsic fluorescence of the aromatic amino acids tryptophan and tyrosine was employed to identify changes in VLP protein tertiary structure as a function of temperature. Upon excitation at 280 nm, fluorescence emission spectra were collected from 300 to 380 nm. Excitation and emission slit widths were set to 3 and 4 nm, respectively.

The fluorescence emission of 8-anilino-1-naphthalene sulphonate (ANS) in the presence of VLPs was utilized as an alternative method to monitor the stability of VLP protein tertiary structure. ANS, a small molecule that is known to have affinity for the apolar regions of proteins, displays weak fluorescence in solution, but, when bound, exhibits enhanced and (usually blue) shifted emission intensities (114). The fluorescence emission spectra of VLP samples prepared with 70 μM ANS were collected from 425 to 550 nm after excitation at 385 nm. While measuring ANS fluorescence, the excitation and emission slit widths were both set to 4 nm.

Another molecular probe, 6-dodecanoyl-2-dimethylaminonaphthalene (laurdan), was used to directly monitor thermally-induced changes in the fluidity of the VLP membrane. The chemical structure of laurdan contains a long acyl chain attached to derivatized naphthalene, thus allowing it to readily incorporate into lipid bilayers. An increase in membrane hydration can drive a transition in bilayer fluidity from a gel (less fluid) to a liquid crystalline (more fluid) phase. When excited at 340 nm, an increase in membrane water content shifts the emission of laurdan from approximately 440 nm to around 490 nm. A useful parameter is the generalized polarization (GP), defined (115) as GP=(I₄₄₀−I₄₈₀)/(I₄₄₀+I₄₈₀), where Iₓ=intensity at wavelength x. Therefore decreasing GP values indicate an increase in membrane fluidity, and vice versa. Slit widths for laurdan experiments were set to 2 nm (excitation) and 5 nm (emission).

1. Intrinsic Fluorescence

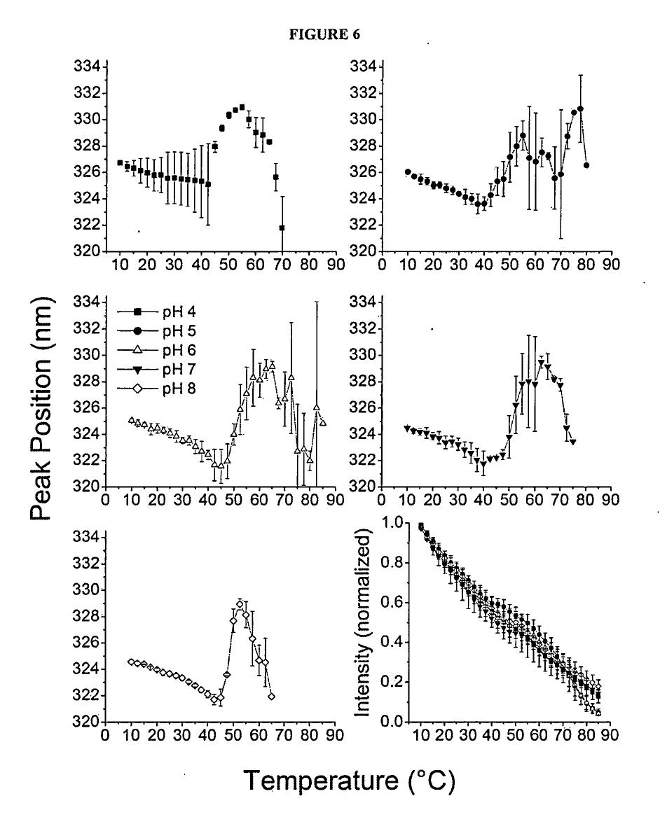

The intrinsic fluorescence emission peak position was determined for all samples as a function of temperature (FIG. 6). In each case, a slight decrease in peak position over the temperature range ˜10-40° C. was followed by a sharp transition to longer wavelengths. For protein samples, a thermally-induced red shift in peak maximum was observed when fluorescent amino acid side chains are exposed to an environment of increased polarity. This was consistent with an unfolding event in which amino acid fluorophores, normally at least partially buried in the apolar protein core, are exposed to the aqueous solvent. At temperatures between 55-65° C. (depending on pH) the peak maximum returns to shorter wavelengths, consistent with the observed aggregation of VLPs at elevated temperatures. The (normalized) emission intensity at 330 nm was also plotted as a function of temperature (FIG. 6). In the absence of structural transitions, such plots typically exhibit a smooth curvilinear decline in emission intensity with increasing temperature due to the intrinsic effect of temperature. Deviations from the curvilinear profile occur for all samples in the range 45-65° C., confirmed that the environments of intrinsic fluorophores were altered upon heating.

2. ANS Fluorescence

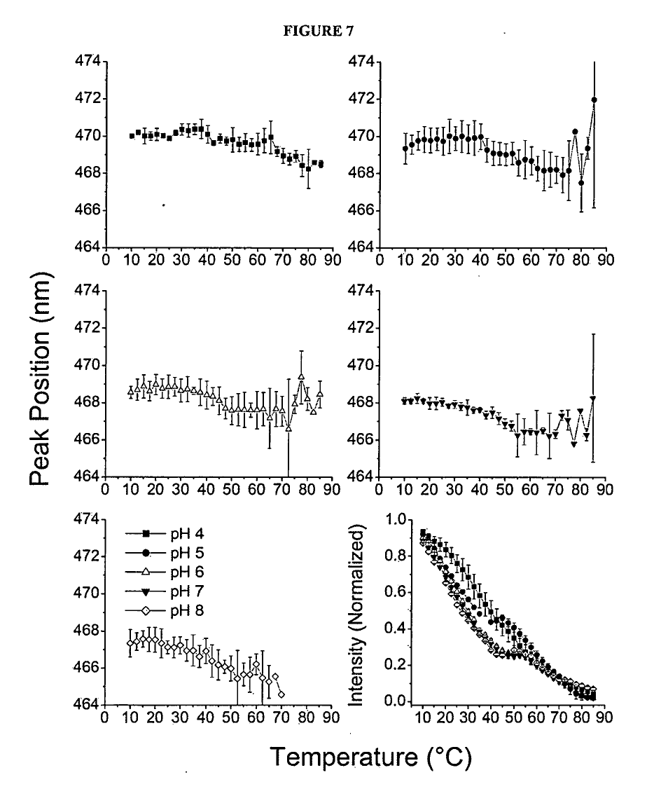

The peak position (≧470 nm) and high intensity of ANS fluorescence emission in the presence of VLPs indicates that ANS was bound to apolar regions of these macromolecular complexes at low temperature, which was expected given the presence of the lipid bilayer. Plots of ANS emission peak position as a function of temperature (FIG. 7) display a temperature-dependent shift to shorter wavelengths in all samples, followed in some cases (i.e. for pH 5, 6, and 7) by a shift to longer wavelengths. In all samples, the extent of change in peak position (2-3 nm) is much less than that observed by intrinsic fluorescence. The high variability of the peak position and noise at elevated temperature make it difficult to draw substantial conclusions from these data. The relative intensity of ANS at 485 nm (FIG. 7) manifested more definite evidence of temperature induced exposure of apolar motifs. This was true in particular for the plots of samples at pH 5-8, in which a slight increase in emission intensity (beginning near 38° C. at pH 5 and 43° C. at pH 6-8) was seen superimposed over the curvilinear decline in emission that corresponded to the expected non-specific thermal quenching of fluorescence.

3. Laurdan Fluorescence

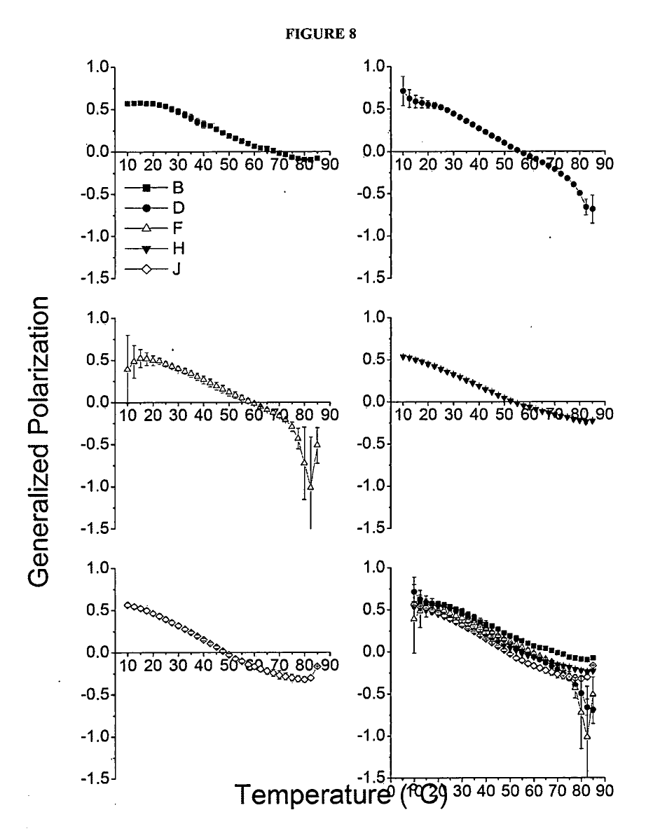

Plots of generalized polarization (GP) as a function of temperature (FIG. 8) indicate a gradual increase in VLP membrane hydration (fluidity) upon heating. Not surprisingly, the pH of VLP suspensions had an effect on the rate and extent of membrane hydration. At low temperatures, the extent of membrane hydration (i.e. GP values) for all samples was very similar. Above 40° C., samples at pH 4 consistently showed the least change in membrane hydration, and, in general, samples prepared at low pH were slower to incorporate water molecules into the bilayer as the temperature is increased (the temperature for which GP=0 increased for more acidic samples). While the GP values of samples prepared at pH 4, 7, or 8 varied in a sigmoidal fashion with temperature, samples at pH 5 and 6 showed a quasi-linear decline in GP over the temperature range examined. The extent of membrane hydration was greatest at high temperature (that is, above 75° C.) for samples at pH 5 and 6. Static light scattering at 340 nm was also monitored during laurdan fluorescence experiments. While these data were similar to the static light scattering measured during the DLS experiments, the transient drop in scattered light intensity observed for samples at pH 4 (FIG. 3(B), 30-35° C.) was not detected during this additional experiment, suggesting that it may have been an artifact.

Empirical Phase Diagram

To create a comprehensive visual representation of VLP physical stability, the data from the various biophysical methods discussed above were converted into a basis set for a multidimensional vector space. An n-dimensional vector was constructed for every combination of temperature and pH for which a measurement was taken (every 2.5° from 10-85° C., unit pH values from 4 to 8), with each vector component a normalized measurement from each of the n techniques applied. The projectors of all vectors in the set were then summed to yield an n×n density matrix with n eigenvectors. The three eigenvectors having the greatest contributions to the data set (i.e. with the greatest eigenvalues) were then used to transform the original n-dimensional vector set into three dimensions. Finally, the three components of each new three dimensional vector were assigned to three different colors (red, green, blue), yielding a unique color combination for each individual vector. By this method, a colored marker could then be assigned to every combination of temperature and pH for which n measurements were taken, yielding a three color map of the entire data set as a function of temperature and pH. The utility of such a diagram is that the most extreme changes in particle structure detected by each technique can be visualized simultaneously as different apparent “phases” of VLP structure. A more detailed description of the generation of EPDs has been presented elsewhere (116, 117).

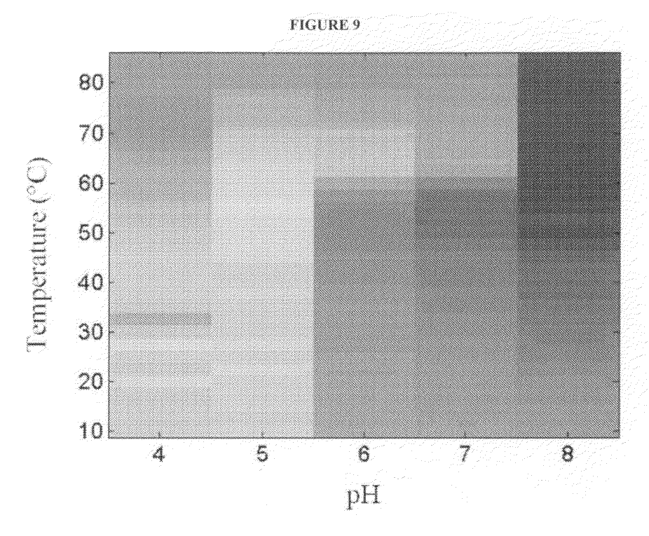

An empirical phase diagram (FIG. 9) was generated from the temperature dependent data presented in the preceding sections. Approximately 10 different phases could be seen over the experimental space, with the largest phase (pH 6-8, low temperature, blue) corresponding to the least structurally disrupted state of the VLPs. There was a transition region that appeared above this phase between 35 and 55° C. for pH 6-7, and from 35 to 50° C. at pH 8 (purple). The variably colored area above 60° C. for pH 6 and 7 corresponded to particle aggregation. The lack of significant aggregation at pH 8 yielded a phase at high temperature (dark red) that was different than that seen at pH 6 or 7. At pH 4 and 5, two different phases were seen at low temperature, both representing significant structural disruption (light blue). Additional temperature-induced conformational changes gave rise to multiple phases above 35° C. in the low pH region (green/orange). The apparent phase boundaries between pH 5 and 6 and above 40° C. represented conditions of intermediate stability, thus providing a starting point for the development of the excipient screening assay of Example 3, below.

### Conclusions

Based upon the combined results of the biophysical characterization of the VLPs, aggregation and changes to solubility are an important factor in the degradation of enveloped virus-based VLPs. The VLPs were the most stable between pH 7 and pH 8. For stability and chemical reasons, a preferred range is between about pH 6.5 and about pH 7.5. Representative buffers that may be used in this range are phosphate, Tris, MES, and citrate.

### Example 3

### Excipient Screening of VLPs

Unless otherwise noted, all potential stabilizers were obtained from Sigma-Aldrich (St. Louis, Mo.). Guanidine HCl, calcium chloride dihydrate, dextrose, D-mannitol, citric acid, and sodium phosphate dibasic were from Fisher Chemical (Fair Lawn, N.J.). Type A porcine gelatin was purchased from Dynagel (Calumet City, Ill.) and D-sucrose and D-trehalose from Ferro-Pfanstiehl Laboratories, Inc. (Waukegan, Ill.). Ectoin (ultra pure) was provided by Bitop AG (Witten, Germany), and NV10 was obtained from Expedeon (formerly Novexin, Cambridge, UK). Concentrated excipient solutions were prepared by dissolution into 20 mM citrate/phosphate (CP) buffer of the appropriate pH. The pH was then adjusted (if necessary) to the target pH using concentrated NaOH or HCl. Final stock solutions were filtered with a 0.22-μm Durapore® (PVDF) membrane syringe filter (Millipore, Billerica, Mass.).

Excipient Screening

The aggregation of VLPs at pH 6 and 60° C. was monitored by measurements of turbidity (optical density at 350 nm, OD₃₅₀) as a function of time. Duplicate samples of VLPs in the presence or absence of various GRAS (generally recognized as safe) agents were prepared at a protein concentration of 55 μg/mL by diluting the concentrated retentate (see above) with 20 mM CP buffer and/or a concentrated excipient solution of the appropriate pH. Measurements were taken every 30 sec over a period of two hours using a temperature-controlled Agilent 8453 spectrophotometer (Palo Alto, Calif.).

The library of GRAS compounds was screened for potential stabilizers of VLPs in solution. Utilizing the empirical phase diagram that was produced from characterization studies of Example 2 (FIG. 9), a screening assay was developed to identify excipients that prevent VLP aggregation (the most apparent physical degradation process). Although the choice of initial temperature and pH conditions for the screening assay was guided by the phase diagram (see preceding Example), the final conditions were optimized to enhance subtle differences between potential stabilizers. Depending on the behavior of the control samples, percent inhibition of aggregation (Table 1) was calculated at either t=15 or t=30 minutes—whichever represented the time of maximal aggregation. The most promising aggregation-inhibiting compounds were found in a variety of molecular classes, including detergents, polyols, amino acids, sugars, and sugar alcohols.

Effect of Individual Stabilizers

Using the top performing aggregation inhibitors from several molecular classes—namely, trehalose, glycerol, sorbitol, lysine, and diethanolamine (given the propensity of detergents to disrupt lipid bilayers, the apparent success of Tween 20 and Brij 35 as aggregation inhibitors may be artifactual)—CD and fluorescence measurements of VLPs in the presence of potential excipients were conducted. The solution pH was set to 7 for these experiments, to more closely approximate an actual vaccine formulation.

As described above, variable-temperature CD measurements were employed to determine if any of the selected compounds stabilize the secondary structure of the VLP proteins (not illustrated). Of all the compounds tested, only sorbitol had a positive effect; the Tₘ of sorbitol-containing samples (55° C.) was slightly elevated relative to the control (54° C.), whereas the other excipients showed Tin values in the range of 50° C. (trehalose) to 53° C. (lysine).

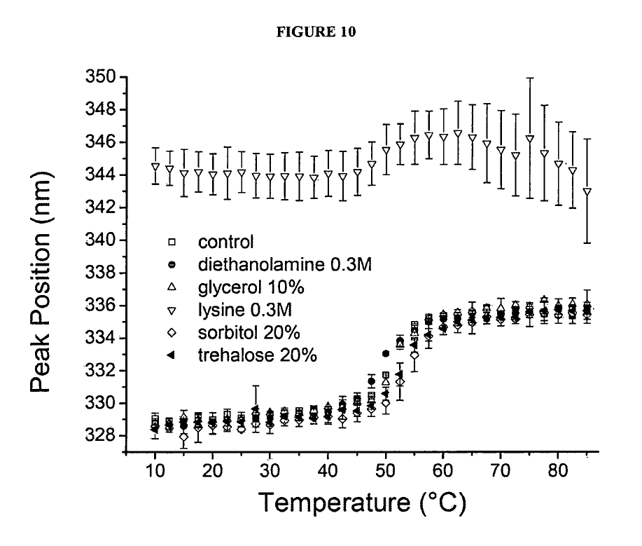

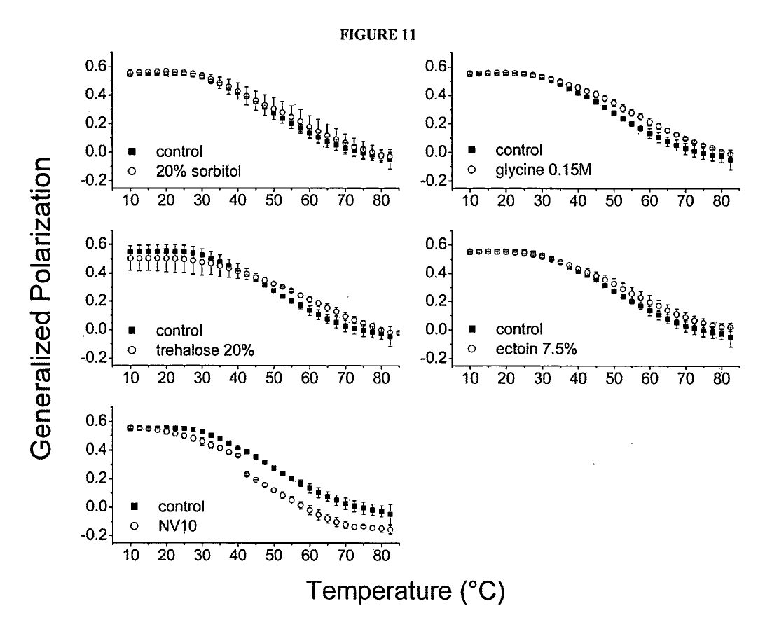

The intrinsic fluorescence method was employed to measure the effect of potential stabilizers on tertiary structure of the VLP proteins. Plots of the emission peak position versus temperature (FIG. 10) show a transition from approximately 329 nm to 336 nm that begins at or near 40° C. for most of the formulations tested. The exception is the formulation containing lysine, which exhibits its fluorescence peak near 344 nm at low temperature and shows evidence of a possible transition to slightly longer wavelengths (+1-2 nm) starting at 43° C. (the error in these measurements prevents the conclusion that the shift is statistically significant). The Tₘ of the control is 51° C. Diethanolamine is not an effective stabilizer, inducing a Tₘ of 49° C., while formulations containing glycerol, trehalose, and sorbitol all show slightly elevated Tₘ values of 52, 53, and 54° C., respectively. Static light scattering collected during these experiments (data not shown) indicated similar behavior among all formulations except the one containing lysine. In the presence of lysine, light scattering intensity was reduced greater than tenfold, suggesting structural disruption of the VLPs. Laurdan fluorescence was used to measure the effect of several compounds on the fluidity of the VLP membrane as a function of increasing temperature. In these experiments, excipients that exhibited weak or no positive effect on physical stability (i.e. diethanolamine, glycerol, and lysine) were supplanted by glycine, ectoin, and NV10. Glycine was introduced due to a personal communication stating that it may stabilize the influenza HA and/or NA proteins. Ectoin (an organic osmolyte) and NV10 (a 5 kDa linear carbohydrate polymer) were tested as potential novel stabilizers of the VLP membrane, based on reports of their general effectiveness in stabilizing macromolecular systems (118). Upon visual inspection of the temperature-dependent GP data (FIG. 11), one or two of the compounds tested appear to inhibit the gel-to-liquid crystal transition. Again using sigmoidal fits to approximate the data, Tₘ values were extracted to quantitatively compare the effects of each stabilizer. As compared to a control sample (Tₘ=52° C.), formulations containing sorbitol or ectoin have slightly higher Tₘ values of 54° C. Given the magnitude of error associated with these measurements, however, the apparent increase in Tₘ is probably not significant in both cases. On the other hand, glycine and trehalose exert a significant stabilizing effect with elevated Tₘ values of 59 and 60° C., respectively. NV10 has a negative effect on the stability of the VLP envelope, inducing (relative to the control) lower GP values (increased membrane hydration) at temperatures above 20° C. The Tₘ calculated for the NV10 formulation is 46° C.

### Conclusions

Several carbohydrates were tested including representative monosaccharides (dextrose, mannitol, sorbitol) and disaccharides (lactose, trehalose, sucrose). In all cases except dextrose, a 10% solution increased aggregation. However, in all cases 20% solutions were effective in inhibiting the aggregation of a VLP solution with trehalose the most effective at 84% inhibition followed closely by sorbitol and lactose). By contrast, oligosaccharides (such as cyclodextrans) were in general not effective in reducing aggregation. Glycerol (a polyalcohol shares structural similarity to carbohydrates) was also effective at reducing (82% inhibition at 10%) aggregation.

Four nonionic surfactants were evaluated at concentrations ranging from 0.01 to 0.10 percent. All four detergents tested (Brij 35, Tween 20, Tween 80 and Plutonic F-68) were effective at inhibiting aggregation. While all detergents tested showed inhibition of aggregation, Tween 20 appears to be superior to the others.

The two commonly used proteins albumin and gelatin that were tested were not effective in inhibiting aggregation, but the results may have been due to issues with the albumin rather than the VLPs.

Representative amino acids were also tested. At 300 mM diethanolamine, arginine and lysine inhibited aggregation by 70%. Guanidine (30%), histidine (30%) and glycine (12%) were less effective and aspartic acid accelerated aggregation. However, it is not clear if the inhibition or acceleration of aggregation was caused directly by the organic compound or indirectly through a change in pH. Further characterization of lysine revealed that use of lysine results in disruption of VLP structure and therefore the reduced turbidity appears to be an artifact and not evidence of inhibition of aggregation.

Representative organic acids were also tested. Ascorbic acid (150 mM) greatly increased aggregation where as similar concentrations of lactic acid and malic acid slightly inhibited aggregation. Again, it is not clear if these observations are a result of a change in pH or a direct interaction of the organic acid with the VLPs.

Several carbohydrates tested show promise for stabilizing enveloped-virus based VLPs. The concentration of the carbohydrate is important with protection afforded by 20% solutions but not 10% solutions or by 15% solutions. But by contrast, 10% glycerol was effective at inhibiting aggregation. Based upon the foregoing, one of skill in the art may readily select a stabilizing amount of these stabilizing agents.

Finally, biophysical studies of trehalose and sorbitol showed that carbohydrates stabilize tertiary structure of viral proteins and trehalose was also shown to slow temperature-induced hydration of lipid bilayer.

Thus, the most promising candidate for the stabilization of VLPs was trehalose at 20% as this carbohydrate was shown to decrease aggregation, stabilize protein tertiary structure and minimize membrane hydration at elevated temperatures. Due to the similarity of structures and concentration dependence of the prevention of aggregation, other carbohydrates are assumed to offer similar protective properties.

### ADDITIONAL REFERENCES

1. Katz, J. M., W. Lim, C. B. Bridges, T. Rowe, J. Hu-Primmer, X. Lu, R. A. Abernathy, M. Clarke, L. Conn, H. Kwong, M. Lee, G. Au, Y. Y. Ho, K. H. Mak, N. J. Cox, and K. Fukuda. 1999. Antibody response in individuals infected with avian influenza A (H5N1) viruses and detection of anti-H5 antibody among household and social contacts. J Infect Dis 180:1763.
2. Peiris, J. S., W. C. Yu, C. W. Leung, C. Y. Cheung, W. F. Ng, J. M. Nicholls, T. K. Ng, K. H. Chan, S. T. Lai, W. L. Lint, K. Y. Yuen, and Y. Guan. 2004. Re-emergence of fatal human influenza A subtype H5N1 disease. Lancet 363:617.
3. Horimoto, T., N. Fukuda, K. Iwatsuki-Horimoto, Y. Guan, W. Lim, M. Peiris, S. Sugii, T. Odagiri, M. Tashiro, and Y. Kawaoka. 2004. Antigenic differences between H5N1 human influenza viruses isolated in 1997 and 2003. J Vet Med Sci 66:303.
4. Tran, T. H., T. L. Nguyen, T. D. Nguyen, T. S. Luong, P. M. Pham, V. C. Nguyen, T. S. Pham, C. D. Vo, T. Q. Le, T. T. Ngo, B. K. Dao, P. P. Le, T. T. Nguyen, T. L. Hoang, V. T. Cao, T. G. Le, D. T. Nguyen, H. N. Le, K. T. Nguyen, H. S. Le, V. T. Le, D. Christiane, T. T. Tran, J. Memo de, C. Schultsz, P. Cheng. W. Lim, P. Horby, and J. Farrar. 2004. Avian influenza A (H5N1) in 10 patients in Vietnam. N Engl J Med 350:1179.
5. Li, K. S., Y. Guan, J. Wang, G. J. Smith, K. M. Xu, L. Duan, A. P. Rahardjo, P. Puthavathana, C. Buranathai, T. D. Nguyen, A. T. Estoepangestie, A. Chaisingh, P. Auewarakul, H. T. Long, N. T. Hanh, R. J. Webby, L. L. Poon, H. Chen, K. F. Shortridge, K. Y. Yuen, R. O. Webster, and J. S. Peiris. 2004. Genesis of a highly pathogenic and potentially pandemic H5N1 influenza virus in eastern Asia. Nature 430:209.
6. Lipatov, A. S., E. A. Govorkova, R. J. Webby, H. Ozaki, M. Peiris, Y. Guan, L. Poon, and R. G. Webster. 2004. Influenza: emergence and control. J Virol 78:8951.
7. Lipatov, A. S., R. J. Webby, E. A. Govorkova, S. Krauss, and R. G. Webster. 2005. Efficacy of H5 influenza vaccines produced by reverse genetics in a lethal mouse model. J Infect Dis 191:1216.
S. Stephenson, I., K. G. Nicholson, J. M. Wood, M. C. Zambon, and J. M. Katz. 2004. Confronting the avian influenza threat: vaccine development for a potential pandemic. Lancet Infect Dis 4:499.
9. Liu, M., J. M. Wood, T. Ellis, S. Krauss, P. Seiler, C. Johnson, E. Hoffmann, J. Humberd, D. Hulse, Y. Zhang, R. G. Webster, and D. R. Perez. 2003. Preparation of a standardized, efficacious agricultural H5N3 vaccine by reverse genetics. Virology 314:580.
10. Subbarao, K., H. Chen, D. Swayne, L. Mingay, E. Fodor, G. Brownlee, X. Xu, X. Lu, J. Katz, N. Cox, and Y. Matsuoka. 2003. Evaluation of a genetically modified reassortant H5N1 influenza A virus vaccine candidate generated by plasmid-based reverse genetics. Virology 305:192.
11. Webby, R. J., D. R. Perez, J. S. Coleman, Y. Guan, J. H. Knight, E. A. Govorkova, L. R. McClain-Moss, J. S. Peiris, J. E. Rehg, E. I. Tuomanen, and R. O. Webster. 2004. Responsiveness to a pandemic alert: use of reverse genetics for rapid development of influenza vaccines. Lancet 363:1099.
12. Treanor, J. J., B. E. Wilkinson, F. Masseoud, J. Hu-Primmer, R. Battaglia, D. O'Brien, M. Wolff, G. Rabinovich, W. Blackwelder, and J. M. Katz. 2001. Safety and immunogenicity of a recombinant hemagglutinin vaccine for H5 influenza in humans. Vaccine 19:1732.
13. Stephenson, I., R. Bugarini, K. G. Nicholson, A. Podda, J. M. Wood, M. C. Zambon, and J. M. Katz. 2005. Cross-reactivity to highly pathogenic avian influenza H5N1 viruses after vaccination with nonadjuvanted and MF59-adjuvanted influenza A/Duck/Singapore/97 (H5N3) vaccine: a potential priming strategy. J Infect Dis 191:1210.
14. Nicholson, K. G., A. E. Colegate, A. Podda, I. Stephenson, J. Wood, E. Ypma, and M. C. Zambon. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937.
15. Subbarao, K., B. R. Murphy, and A. S. Fauci. 2006. Development of effective vaccines against pandemic influenza. Immunity 24:5.
16. Kuper, C. F., P. J. Koornstra, D. M. Hameleers, J. Biewenga, B. J. Spit, A. M. Duijvestijn, P. J. van Breda Vriesman, and T. Sminia. 1992. The role of nasopharyngeal lymphoid tissue. Immunol Today 13:219.
17. Liang, B., L. Hyland, and S. Hon. 2001. Nasal-associated lymphoid tissue is a site of long-term virus-specific antibody production following respiratory virus infection of mice. J Virol 75:5416.
18. Zuercher, A. W., S. E. Coffin, M. C. Thurnheer, P. Fundova, and J. J. Cebra. 2002. Nasal-associated lymphoid tissue is a mucosal inductive site for virus-specific humoral and cellular immune responses. J Immunol 168:1796.
19. Brandtzaeg, P. 1989. Overview of the mucosal immune system. Curr Top Microbiol Immunol 146:13.
20. Takada, A., S. Matsushita, A. Ninomiya, Y. Kawaoka, and H. Kida. 2003. Intranasal immunization with formalin-inactivated virus vaccine induces a broad spectrum of heterosubtypic immunity against influenza A virus infection in mice. Vaccine 21:3212.
21. Tamura, S. I., H. Asanuma, Y. Ito, Y. Hirabayashi, Y. Suzuki, T. Nagamine, C. Aizawa, T. Kurata, and A. Oya. 1992. Superior cross-protective effect of nasal vaccination to subcutaneous inoculation with influenza hemagglutinin vaccine. Eur J Immunol 22:477.
22. Tumpey, T. M., M. Renshaw, J. D. Clements, and J. M. Katz. 2001. Mucosal delivery of inactivated influenza vaccine induces B-cell-dependent heterosubtypic cross-protection against lethal influenza A H5N1 virus infection. J Virol 75:5141.
23. Kang, S. M., L. Guo, Q. Yao, I. Skountzou, and R. W. Compans. 2004. Intranasal immunization with inactivated influenza virus enhances immune responses to coadministered simian-human immunodeficiency virus-like particle antigens. J Virol 78:9624.
24. Guo, L., X. Lu, S. M. Kang, C. Chen, R. W. Compans, and Q. Yao. 2003. Enhancement of mucosal immune responses by chimeric influenza HA/SHIV virus-like particles. Virology 313:502.
25. Yao, Q., R. Zhang, L. Guo, M. Li, and C. Chen. 2004. Th cell-independent immune responses to chimeric hemagglutinin/simian human immunodeficiency virus-like particles vaccine. J Immunol 173:1951.
26. Latham, T., and J. M. Galarza. 2001. Formation of wild-type and chimeric influenza virus-like particles following simultaneous expression of only four structural proteins. J Virol 75:6154.
27. Galarza, J. M., T. Latham, and A. Cupo. 2005. Virus-like particle vaccine conferred complete protection against a lethal influenza virus challenge. Viral Immunol 18:365.
28. Fromantin, C., B. Jamot, J. Cohen, L. Piroth, P. Pothier, and E. Kohli. 2001. Rotavirus 2/6 virus-like particles administered intranasally in mice, with or without the mucosal adjuvants cholera toxin and Escherichia coli heat-labile toxin, induce a Th1/Th2-like immune response. J Virol 75:11010.
29. Harrington, P. R., B. Yount, R. E. Johnston, N. Davis, C. Moe, and R. S. Baric. 2002. Systemic, mucosal, and heterotypic immune induction in mice inoculated with Venezuelan equine encephalitis replicons expressing Norwalk virus-like particles. J Virol 76:730.
30. Shi, W., J. Liu, Y. Huang, and L. Qiao. 2001. Papillomavirus pseudovirus: a novel vaccine to induce mucosal and systemic cytotoxic T-lymphocyte responses. J Virol 75:10139.
31. Han, M. G., S. Cheetham, M. Azevedo, C. Thomas, and L. J. Saif 2006. Immune responses to bovine norovirus-like particles with various adjuvants and analysis of protection in gnotobiotic calves. Vaccine 24:317.
32. Illum, L. 1998. Chitosan and its use as a pharmaceutical excipient. Pharm Res 15:1326.
33. Illum, L., I. Jabbal-Gill, M. Hinchcliffe, A. N. Fisher, and S. S. Davis. 2001. Chitosan as a novel nasal delivery system for vaccines. Adv Drug Deliv Rev 51:81.
34. Soane, R. J., M. Hinchcliffe, S. S. Davis, and L. Illum. 2001. Clearance characteristics of chitosan based formulations in the sheep nasal cavity. Int J Pharm 217:183.
35. Baudner, B. C., M. M. Giuliani, J. C. Verhoef, R. Rappuoli, H. E. Junginger, and G. D. Giudice. 2003. The concomitant use of the LTK63 mucosal adjuvant and of chitosan-based delivery system enhances the immunogenicity and efficacy of intranasally administered vaccines. Vaccine 21:3837.
36. Fujihashi, K., T. Koga, F. W. van Ginkel, Y. Hagiwara, and J. R. McGhee. 2002. A dilemma for mucosal vaccination: efficacy versus toxicity using enterotoxin-based adjuvants. Vaccine 20:2431.
37. Mutsch. M., W. Zhou, P. Rhodes, M. Bopp, R. T. Chen, T. Under, C. Spyr, and R. Steffen. 2004. Use of the inactivated intranasal influenza vaccine and the risk of Bell's palsy in Switzerland. N Engl J Med 350:896.
38. Baldridge, J. R., Y. Yorgensen, J. R. Ward, and J. T. Ulrich. 2000. Monophosphoryl lipid A enhances mucosal and systemic immunity to vaccine antigens following intranasal administration. Vaccine 18:2416.
39. Baldrick, P., D. Richardson, G. Elliott, and A. W. Wheeler. 2002. Safety evaluation of monophosphoryl lipid A (MPL): an immunostimulatory adjuvant. Regul Toxicol Pharmacol 35:398.
40. Baldridge, S. R., P. McGowan, S. T. Evans, C. Cluff, S. Mossman, D. Johnson, and D. Persing. 2004. Taking a Toll on human disease: Toll-like receptor 4 agonists as vaccine adjuvants and monotherapeutic agents. Expert Opin Biol Ther 4:1129.
41. Baldridge, J. GlaxoSmithKline, Personal Communication.
42. Huang, J., R. J. Garmise, T. M. Crowder, K. Mar, C. R. Hwang, A. J. Hickey, J. A. Mikszta, and V. J. Sullivan. 2004. A novel dry powder influenza vaccine and intranasal delivery technology: induction of systemic and mucosal immune responses in rats. Vaccine 23:794.
43. Mills, K. H., C. Cosgrove, E. A. McNeela, A. Sexton, R. Giemza, I. Jabbal-Gill, A. Church, W. Lin, L. Illum, A. Podda, R. Rappuoli, M. Pizza, G. E. Griffin, and D. J. Lewis. 2003. Protective levels of diphtheria-neutralizing antibody induced in healthy volunteers by unilateral priming-boosting intranasal immunization associated with restricted ipsilateral mucosal secretory immunoglobulin a, Infect Immun 71:726.
44. Wimer-Mackin, S., M. Hinchcliffe, C. R. Petrie, S. J. Warwood, W. T. Tino, M. S. Williams, J. P. Stenz, A. Cheff, and C. Richardson. 2006. An intranasal vaccine targeting both the Bacillus anthracis toxin and bacterium provides protection against aerosol spore challenge in rabbits. Vaccine in press.
45. Noad, R., and P. Roy. 2003. Virus-like particles as inummogens. Trends Microbial 11:438.
46. Yao, Q., V. Vuong, M. Li, and R. W. Compans. 2002. Intranasal immunization with SIV virus-like particles (VLPs) elicits systemic and mucosal immunity. Vaccine 20:2537.
47. Pushko, P., T. M. Tumpey, F. Bu, S. Knell, R. Robinson, and G. Smith. 2005. Influenza virus-like particles comprised of the HA, NA, and M1 proteins of H9N2 influenza virus induce protective immune responses in BALB/c mice. Vaccine 23:5751,
48. Baumert, T. F., S. Ito, D. T. Wong, and T. J. Liang. 1998. Hepatitis C virus structural proteins assemble into viruslike particles in insect cells. J Virol 72:3827.
49. Yao, Q., Z. Bu, A. Vzorov, C. Yang, and R. W. Compans. 2003. Virus-like particle and DNA-based candidate AIDS vaccines. Vaccine 21:638.
50. Tacket, C. O., M. B. Sztein, G. A. Losonsky, S. S. Wasserman, and M. K. Estes. 2003. Humoral, mucosal, and cellular immune responses to oral Norwalk virus-like particles in volunteers. Clin Immunol 108:241.
51. Gomez-Puertas, P., C. Albo, E. Perez-Pastrana, A. Vivo, and A. Portela. 2000. Influenza virus matrix protein is the major driving force in virus budding. J Virol 74:11538.
52. Gheysen, D., E. Jacobs, F. de Foresta, C. Thiriart, M. Francotte, D. Thines, and M. De Wilde. 1989. Assembly and release of HIV-1 precursor Pr55gag virus-like particles from recombinant baculovirus-infected insect cells. Cell 59:103.
53. Johnson, M. C., H. M. Scobie, and V. M. Vogt. 2001. PR domain of rous sarcoma virus Gag causes an assembly/budding defect in insect cells. J Virol 75:4407.
54. Kakker, N. K., M. V. Mikhailov, M. V. Nermut, A. Burny, and P. Roy. 1999. Bovine leukemia virus Gag particle assembly in insect cells: formation of chimeric particles by domain-switched leukemia/lentivirus Gag polyprotein. Virology 265:308.
55. Luo, L., Y. Li, and C. Y. Kang. 1990. Expression of gag precursor protein and secretion of virus-like gag particles of HIV-2 from recombinant baculovirus-infected insect cells. Virology 179:874.
56. Morikawa, S., T. F. Booth, and D. H. Bishop. 1991. Analyses of the requirements for the synthesis of virus-like particles by feline immunodeficiency virus gag using baculovirus vectors. Virology 183:288.
57. Takahashi, R. H., K. Nagashima, T. Kurata, and H. Takahashi. 1999. Analysis of human lymphotropic T-cell virus type II-like particle production by recombinant baculovirus-infected insect cells. Virology 256:371.
58. Yamshchikov, G. V., G. D. Ritter, M. Vey, and R. W. Compans. 1995. Assembly of SIV virus-like particles containing envelope proteins using a baculovirus expression system. Virology 214:50.
59. Weldon, R. A., Jr., C. R. Erdie, M. G. Oliver, and J. W. Wills. 1990. Incorporation of chimeric gag protein into retroviral particles. J Virol 64:4169.
60. Andrawiss, M., Y. Takeuchi, L. Hewlett, and M. Collins. 2003. Murine leukemia virus particle assembly quantitated by fluorescence microscopy: role of Gag-Gag interactions and membrane association. J Virol 77:11651.
61. Leser, G. P., and R. A. Lamb. 2005. Influenza virus assembly and budding in raft-derived microdomains: a quantitative analysis of the surface distribution of HA, NA and M2 proteins. Virology 342:215.
62. Takeda, M., G. P. Leser, C. J. Russell, and R. A. Lamb. 2003. Influenza virus hemagglutinin concentrates in lipid raft microdomains for efficient viral fusion. Proc Natl Acad Sci USA 100:14610.
63. Campbell, S. M., S. M. Crowe, and J. Mak. 2001. Lipid rafts and HIV-1: from viral entry to assembly of progeny vinous. J Clin Virol 22:217.
64. Sandrin, V., and F. L. Cosset. 2006. Intracellular versus cell surface assembly of retroviral pseudotypes is determined by the cellular localization of the viral glycoprotein, its capacity to interact with Gag, and the expression of the Nef protein. J Biol Chem 281:528.
65. Salazar-Gonzalez, R. M., and S. J. McSorley. 2005. Salmonella flagellin, a microbial target of the innate and adaptive immune system. Immunol Lett 101:117.
66. Cuadros, C., F. J. Lopez-Hernandez, A. L. Dominguez, M. McClelland, and J. Lustgarten. 2004. Flagellin fusion proteins as adjuvants or vaccines induce specific immune responses. Infect Immun 72:2810.
67. Didierlaurent, A., I. Ferrero, L. A. Otten, B. Dubois, M. Reinhardt, H. Carlsen, R. Blomhoff, S. Akira, J. P. Kraehenbuhl, and J. C. Sirard. 2004. Flagellin promotes myeloid differentiation factor 88-dependent development of Th2-type response. J Immunol 172:6922.
68. Tsujimoto, H., T. Uchida, P. A. Efron, P. O. Scumpia A. Verma, T. Matsumoto, S. K. Tschoeke, R. F. Ungaro, S. Ono, S. Seki, M. J. Clare-Salzler, H. V. Baker, H. Mochizuki, R. Ramphal, and L. L. Moldawer. 2005. Flagellin enhances NK cell proliferation and activation directly and through dendritic cell-NK cell interactions. J Leukoc Biol 78:888.
69. Hayashi, F., T. K. Means, and A. D. Luster. 2003. Toll-like receptors stimulate human neutrophil function. Blood 102:2660.
70. Hayashi, F., K. D. Smith, A. Ozinsky, T. R. Hawn, E. C. Yi, D. R. Goodlett, J. K. Eng, S. Akira, D. M. Underhill, and A. Aderem. 2001. The innate immune response to bacterial flagellin is mediated by Toll-like receptor 5. Nature 410:1099.
71. Gewirtz, A. T., P. O. Simon, Jr., C. K. Schmitt, L. J. Taylor, C. H. Hagedorn, A. D. O'Brien, A. S. Neish, and J. L. Madam. 2001. Salmonella typhimurium translocates flagellin across intestinal epithelia, inducing a proinflammatory response. J Clin Invest 107:99.
72. Means, T. K., F. Hayashi, K. D. Smith, A. Aderem, and A. D. Luster. 2003. The Toll-like receptor 5 stimulus bacterial flagellin induces maturation and chemokine production in human dendritic cells. J Immunol 170:5165.
73. Smith, K. D., E. Andersen-Nissen, F. Hayashi, K. Strobe, M. A. Bergman, S. L. Barrett, B. T. Cookson, and A. Aderem. 2003. Toll-like receptor 5 recognizes a conserved site on flagellin required for protofilament formation and bacterial motility. Nat Immunol 4:1247.
74. Honko, A. N., N. Sriranganathan, C. J. Lees, and S. B. Mizel. 2006. Flagellin is an effective adjuvant for immunization against lethal respiratory challenge with Yersinia pestis. Infect Immun 74:1113.
75. Jeon, S. H., T. Ben-Yedidia, and R. Arnon. 2002. Intranasal immunization with synthetic recombinant vaccine containing multiple epitopes of influenza virus. Vaccine 20:2772.
76. Levi, R., and R. Arnon. 1996. Synthetic recombinant influenza vaccine induces efficient long-term immunity and cross-strain protection. Vaccine 14:85.
77. Lee, S. E., S. Y. Kim, B. C. Jeong, Y. R. Kim, S. J. Bae, O, S. Aim, J. J. Lee, H. C. Song, J. M. Kim, H. E. Choy, S. S. Chung, M. N. Kweon, and J. H. Rhee. 2006. A bacterial flagellin, Vibrio vulnificus FlaB, has a strong mucosal adjuvant activity to induce protective immunity. Infect Immun 74:694.
78. Applequist, S. E., E. Roliman, M. D. Wareing, M. Liden, B. Rozell, J. Hinkula, and H. G. Ljunggren. 2005. Activation of innate immunity, inflammation, and potentiation of DNA vaccination through mammalian expression of the TLR5 agonist flagellin. J Immunol 175:3882.
79. Ramos, H. C., M. Rumbo, and J. C. Sirard. 2004. Bacterial flagellins: mediators of pathogenicity and host immune responses in mucosa. Trends Microbiol 12:509.
80. Holsinger, L. J., and R. A. Lamb. 1991. Influenza virus M2 integral membrane protein is a homotetramer stabilized by formation of disulfide bonds. Virology 183:32.
81. Lamb, R. A., S. L. Zebedee, and C. D. Richardson. 1985. Influenza virus M2 protein is an integral membrane protein expressed on the infected-cell surface. Cell 40:627.
82. Holsinger, L. J., D. Nichani, L. H. Pinto, and R. A. Lamb. 1994. Influenza A virus M2 ion channel protein: a structure-function analysis. J Virol 68:1551.
83. Takeda, M., A. Pekosz, K. Shuck, L. H. Pinto, and R. A. Lamb. 2002. Influenza a virus M2 ion channel activity is essential for efficient replication in tissue culture. J Virol 76:1391.
84. Frace, A. M., A. I. Klimov, T. Rowe, R. A. Black, and J. M. Katz. 1999. Modified M2 proteins produce heterotypic immunity against influenza A virus. Vaccine 17:2237.
85. Neirynck, S., T. Demo, X. Saelens, P. Vanlandschoot, W. M. Jou, and W. Fiers. 1999. A universal influenza A vaccine based on the extracellular domain of the M2 protein. Nat Med 5:1157.
86. Slepushkin, V. A., J. M. Katz, R. A. Black, W. C. Gamble, P. A. Rota, and N. J. Cox. 1995. Protection of mice against influenza A virus challenge by vaccination with baculovirus-expressed M2 protein. Vaccine 13:1399.
87. Treanor, I. J., E. L. Tierney, S. L. Zebedee, R. A. Lamb, and B. R. Murphy. 1990. Passively transferred monoclonal antibody to the M2 protein inhibits influenza A virus replication in mice. J Virol 64:1375.
88. De Filette, M., W. Min Jou, A. Birkett, K. Lyons, B. Schultz, A. Tonkyro, S. Resch, and W. Fiers. 2005. Universal influenza A vaccine: optimization of M2-based constructs. Virology 337:149.
89. De Filette, M., A. Ramne, A. Birkett, N. Lycke, B. Lowenadler, W. Min Jou, X. Saelens, and W. Fiers. 2006. The universal influenza vaccine M2e-HBc administered intranasally in combination with the adjuvant CTA1-DD provides complete protection. Vaccine 24:544.
90. Fiers, W., M. De Filette, A. Birkett, S. Neirynck, and W. Min Jou. 2004. A “universal” human influenza A vaccine. Virus Res 103:173.
91. Liu, W., Z. Peng, Z. Liu, Y. Lu, J. Ding, and Y. H. Chen. 2004. High epitope density in a single recombinant protein molecule of the extracellular domain of influenza. A virus M2 protein significantly enhances protective immunity. Vaccine 23:366.
92. Fan, J., X. Liang, M. S. Horton, H. C. Perry, M. P. Citron, G. J. Heidecker, T. M. Fu, J. Joyce, C. T. Przysiecki, P. M. Keller, V. M. Garsky, R. Ionescu, Y. Rippeon, L. Shi, M. A. Chastain, J. H. Condra, M. E. Davies, J. Liao, E. A. Emini, and J. W. Shiver. 2004. Preclinical study of influenza virus A M2 peptide conjugate vaccines in mice, ferrets, and rhesus monkeys. Vaccine 22:2993.
93. Iouescu, R. M., C. T. Przysiecki, X. Liang, V. M. Garsky, J. Fan, B. Wang, R. Troutman, Y. Rippeon, E. Flanagan, J. Shiver, and L. Shi. 2006. Pharmaceutical and immunological evaluation of human papillomavims viruslike particle as an antigen carrier. J Pharm Sci 95:70.
94. Hatziioannou, T., E. Delahaye, F. Martin, S. J. Russell, and F. L. Cosset. 1999. Retroviral display of functional binding domains fused to the amino terminus of influenza hemagglutinin. Hum Gene Ther 10:1533.
95. Li, Z. N., S. N. Mueller, L. Ye, Z. Bu, C. Yang, R. Ahmed, and D. A. Steinhauer. 2005. Chimeric influenza virus hemagglutinin proteins containing large domains of the Bacillus anthracis protective antigen: protein characterization, incorporation into infectious influenza viruses, and antigenicity. J Virol 79:10003.
96. Haynes, J. R., S. X. Cao, B. Rovinski, C. Sia, O. James, G. A. Dekaban, and M. H. Klein. 1991. Production of immunogenic HIV-1 viruslike particles in stably engineered monkey cell lines. AIDS Res Hum Retroviruses 7:17.
97. Rovinski, B., J. R. Haynes, S. X. Cao, O. James, C. Sia, S. Zolla-Pazner, T. J. Matthews, and M. H. Klein. 1992. Expression and characterization of genetically engineered human immunodeficiency virus-like particles containing modified envelope glycoproteins: implications for development of a cross-protective AIDS vaccine. J Virol 66:4003.
98. Fyuan, E. F., R. G. Webster, D. H. Fuller, J. R. Haynes, J. C. Santoro, and H. L. Robinson. 1995. DNA vaccines: a novel approach to immunization. Int J Immunopharmacol 17:79.
99. Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proc Natl Acad Sci USA 90:11478.
100. Kodihalli, S., J. R. Haynes, H. L. Robinson, and R. G. Webster. 1997. Cross-protection among lethal H5N2 influenza viruses induced by DNA vaccine to the hemagglutinin. J Virol 71:3391.
101. Robinson, H. L., S. Lu, D. M. Feltquate, C. T. Torres, J. Richmond, C. M. Boyle, M. J. Morin, J. C. Santoro, R. G. Webster, D. Montefiori, Y. Yasutomi, N. L. Levin, K. Manson, M. Wyand, and J. R. Haynes. 1996. DNA vaccines. AIDS Res Hum Retroviruses 12:455.
102. Drape, R. J., M. D. Macklin, L. J. Barr, S. Jones, J. R. Haynes, and H. J. Dean. 2006. Epidermal DNA vaccine for influenza is immunogenic in humans. Vaccine in press.
103. Kretzchmar, E., R. Geyer, and H. D. Klenk. 1994. Baculovirus infection does not alter N-glycosylation in Spodoptera frugiperda cells. Biol Chem Hoppe Seyler 375:23.
104. Lu, D., and A. J. Hickey. 2005. Liposomal dry powders as aerosols for pulmonary delivery of proteins. AAPS Pharm SciTech 6:E641.
105. Cowdery, S., M. Frey, S. Orlowski, and A. Gray. 1976. Stability characteristics of freeze-dried human live virus vaccines. Dev Biol Stand 36:297.
106. Peetermans, J., G. Colinet, A. Bouillet, E. D'Hondt, and J. Stephenne. 1976. Stability of live, freeze-dried virus vaccines. Dev Biol Stand 36:291.
107. Yannarell, D. A., K. M. Goldberg, and R. N. Hjorth. 2002. Stabilizing cold-adapted influenza virus vaccine under various storage conditions. J Virol Methods 102:15.
108. Sampson, H. A., J. Bernhisel-Broadbent, E. Yang, and S. M. Scanlon. 1991. Safety of casein hydrolysate formula in children with cow milk allergy. J Pediatr 118:520.
109. Gambaryan A, A. Tuzikov. G. Pazynina, N. Bovin, A. Balish, and A. Klimov. 2005. Evolution of the receptor binding phenotype of influenza A (H5) viruses. Virology (electronic publication ahead of print version).
110. Suzuki, Y. 2005. Sialobiology of Influenza: Molecular Mechanism of Host Range Variation of Influenza Viruses. Biological and Pharmaceutical Bulletin 28:399-408.
111. Murphy, B. R., R. G. Webster. 1996. Orthomyxoviruses. In: Fields B N, Knipe D M, Howley P M, et al., eds. Fields virology. 3rd ed. Philadelphia, Pa.: Lippincott-Raven Publishers, 1397-445.
112. Clements M. L., R. F. Betts, E. L. Tierney, B. R. Murphy. 1986. Serum and nasal wash antibodies associated with resistance to experimental challenge with influenza A wild-type virus. J Clin Microbiol 24:157-60.
113. Couch R. B., J. A. Kasel. 1983. Immunity to influenza in man. Ann Rev Microbiol 37:529-49.
114. Lakowicz J R. 1999. Principles of fluorescence spectroscopy. New York: Kluwer Academic/Plenum. 698 p.
115. Parasassi T, De Stasio G, Ravagnan G, Rusch R M, Gratton E. 1991. Quantitation of lipid phases in phospholipid vesicles by the generalized polarization of Laurdan fluorescence. Biophys J 60: 179-189.
116. Kueltzo L A, Ersoy B, Ralston J P, Middaugh C R. 2003. Derivative absorbance spectroscopy and protein phase diagrams as tools for comprehensive protein characterization: a bGCSF case study. J Pharm Sci 92: 1805-1820.
117. Fan H, Ralston J, Dibiase M, Faulkner E, Middaugh C R. 2005. Solution behavior of IFN-beta-1a: an empirical phase diagram based approach. J Pharm Sci 94: 1893-1911.
118. Borges N, Ramos A, Raven N D, Sharp R J, Santos H. 2002. Comparative study of the thermostabilizing properties of mannosylglycerate and other compatible solutes on model enzymes. Extremophiles 6: 209-216.

1. A method for stabilizing a solution containing an influenza antigen enveloped virus-based virus-like particle preparation comprising:
(a) providing the solution containing the influenza antigen enveloped virus-based virus-like particle; and
(b) (1) adding a stabilizing amount of a stabilizing agent selected from a monosaccharide, sorbitol, a disaccharide, trehalose, diethanolamine, glycerol, glycine, and a combination of the preceding stabilizing agents to influenza antigen enveloped virus-based virus-like particle preparation, (2) buffering the solution so that the pH is between about pH 6.5 and about pH 8.0, between about pH 6.5 and about pH 7.5, or about pH 7, or (3) both steps (1) and (2),
wherein the influenza antigen enveloped virus-based virus-like particle preparation after step (b) exhibits at least one of the following characteristics (i) reduced aggregation of the virus-like particles as compared to the influenza antigen enveloped virus-based virus-like particle preparation before step (b) as measured by optical density, (ii) stabilized influenza antigen as compared to the influenza antigen enveloped virus-based virus-like particle preparation before step (b) as measured by circular dichroism or ANS binding, and (iii) reduced temperature induced hydration of the lipid bilayer of the virus-like particle as compared to the influenza antigen enveloped virus-based virus-like particle preparation before step (b) as measured by laurdan fluorescence.

2. The method of claim 1, wherein the buffering is performed using a buffering agent selected from the group consisting of phosphate, Tris, MES, citrate and other GRAS buffers.

3. The method of claim 1, wherein the stabilizing agent is selected from trehalose, sorbitol, diethanolamine, glycerol, glycine and a combination of the preceding stabilizing agents and the characteristic is (i).

4. The method of claim 1, wherein the stabilizing agent is selected from trehalose, sorbitol, and a combination of the preceding stabilizing agents and the characteristic is (ii).

5. The method of claim 1, wherein the stabilizing agent is selected from trehalose and glycine and the characteristic is (iii).

6. The method of claim 1, wherein the stabilizing agent is trehalose and all three characteristics are present.

7. The method of claim 1, wherein the influenza antigen enveloped virus-based virus-like particle comprises a hemagglutinin polypeptide.

8. The method of claim 1, wherein the influenza antigen enveloped virus-based virus-like particle comprises a second polypeptide selected from the group comprising a gag polypeptide, an influenza M1 polypeptide, a Newcastle disease virus matrix polypeptide, an Ebola virus VP40 polypeptide and a Marburg virus VP40 polypeptide.

9. The method of claim 8, wherein said gag polypeptide is from a retrovirus selected from the group consisting of: murine leukemia virus, human immunodeficiency virus, Alpharetroviruses, Betaretroviruses, Gammaretroviruses, Deltaretroviruses and Lentiviruses.

10. The method of claim 8, wherein said gag polypeptide is from a murine leukemia virus.

11. The method of claim 1, wherein the influenza antigen enveloped virus-based virus-like particle further comprises a neuraminidase polypeptide.

12. The method of claim 1, wherein the stabilizing agent is selected from monosaccharide, sorbitol, a disaccharide, and trehalose, and the stabilizing amount is greater than 10% (w/w) or at least about 20% (w/w).

13. The method of claim 1, wherein the stabilizing does not require glass formation.

14. The method of claim 1, wherein the stabilizing amount is less that the amount required for glass formation upon freezing.

15. The method of claim 1, wherein the stabilizing agent is not sucrose.

16. The method of claim 1, wherein the influenza antigen enveloped virus-based virus-like particle preparation further comprises an adjuvant in admixture with the influenza antigen enveloped virus-based virus-like particle.

17-21. (canceled)

22. The method claim 1, further comprising (c) storing the solution in liquid form for a period of time of at least two weeks, at least one month, at least two months, at least three months, at least four months, at least six months, or at least one year, wherein the influenza antigen enveloped virus-based virus-like particle preparation after such time period induces at least eighty percent, at least ninety percent, or at least ninety five percent of the immune response induced by the influenza antigen enveloped virus-based virus-like particle preparation before such time period.

23. An influenza antigen enveloped virus-based virus-like particle preparation comprising influenza antigen enveloped virus-based virus-like particles and a stabilizing amount of a stabilizing agent selected from trehalose, sorbitol, diethanolamine, glycerol, glycine, and a combination of the preceding stabilizing agents to influenza antigen enveloped virus-based virus-like particle preparation, wherein the influenza antigen enveloped virus-based virus-like particle preparation exhibits at least one of the following characteristics (i) reduced aggregation of the virus-like particles as compared to a influenza antigen enveloped virus-based virus-like particle preparation without the stabilizing agent as measured by optical density, (ii) stabilized influenza antigen as compared to the influenza antigen enveloped virus-based virus-like particle preparation without the stabilizing agent as measured by circular dichroism or ANS binding, and (iii) reduced temperature induced hydration of the lipid bilayer of the virus-like particle as compared to the influenza antigen enveloped virus-based virus-like particle preparation without the stabilizing agent as measured by laurdan fluorescence.

24-43. (canceled)

44. A method for treating or preventing influenza comprising administering to a subject an immunogenic amount of a solution containing an immunogenic amount of an influenza antigen enveloped virus-based virus-like particle preparation stabilized in accordance with claim 1.

45. The method of claim 44, wherein the administering induces a protective immunization response in the subject.

46. (canceled)


SEQUENCE LISTING (5 sequences)

SEQ ID NO: 1 - DNA, 20 bp, Artificial Sequence
  Synthetic Construct
tccatgacgttcctgacgtt

SEQ ID NO: 2 - DNA, 18 bp, Artificial Sequence
  Synthetic Construct
tctcccagcgtgcgccat

SEQ ID NO: 3 - DNA, 30 bp, Artificial Sequence
  Synthetic Construct
accgatgacgtcgccggtgacggcaccacg

SEQ ID NO: 4 - DNA, 24 bp, Artificial Sequence
  Synthetic Construct
tcgtcgttttgtcgttttgtcgtt

SEQ ID NO: 5 - DNA, 20 bp, Artificial Sequence
  Synthetic Construct
tccatgacgttcctgatgct